(19) United States
(12) Patent Application Publication
(10) Pub. No.: US 2024/0077471 A1
(43) Pub. Date: Mar. 7, 2024

(54) Methods and Compositions for Diagnosing and Treating Rare Genetic DIseases

(71) Applicant: Rosebud Biosciences, Inc., Orinda, CA (US)

(72) Inventor: Kitch Wilson, Orinda, CA (US)

(73) Assignee: Rosebud Biosciences, Inc., Orinda, CA (US)

(21) Appl. No.: 18/260,220

(22) PCT Filed: Jan. 28, 2022
(86) PCT No.: PCT/US2022/014243

§ 371 Date: Jun. 30, 2023

Related U.S. Application Data
(60) Provisional application No. 63/144,818, filed on Feb. 2, 2021.

(51) Int. Cl. G01N 33/50; C12N 5/077; C12N 5/0793; C12Q 1/6883

(52) U.S. Cl. CPC G01N 33/5073; C12N 5/0619; C12N 5/0657; C12Q 1/6883; C12N 2506/45; C12Q 2600/156

(57) ABSTRACT

Collectively, rare diseases affect approximately 350 million people worldwide, of which half are diagnosed in childhood. Even though more than 80% of rare diseases are genetic in origin and begin in utero, drug target discovery has historically been stymied due to difficulties in obtaining fetal, neonatal, and pediatric tissues for genetic study and drug discovery. Disease modeling with patient-specific hiPSCs is an ideal platform for both rapid identification and experimental validation of early life disease mechanisms. This invention combines high content genomic assays, phenotypic measurements, and machine learning (Artificial Intelligence/Machine Learning) with hiPSC disease models to identify DNA mutations causing early life diseases.

### BACKGROUND

There are approximately 7,000 known rare diseases (defined in the U.S. as fewer than 200,000 affected). This includes pediatric cardiomyopathies, lysosomal storage diseases, muscular dystrophies, cystic fibrosis, Angel-man, Rett and Prader Willi syndromes, and thousands more. Although rare individually, collectively rare diseases affect approximately 350 million people worldwide, of which 50-75% are diagnosed in childhood. Greater than 80% of rare diseases are genetic in origin and begin in utero, but unfortunately 95% still lack treatment. This is an enormous problem as 30% of children with these rare diseases will not live to see their 5^{th} birthday.

Recent drug development has increased the availability of treatments for young patients, but the majority of FDA-approved rare disease pediatric drugs are merely re-purposed existing drugs and target a limited number of diseases. Novel drug discovery for rare diseases of childhood has been stymied by difficulties in:
obtaining fetal, neonatal and pediatric tissues for genetic study,
challenges in conducting adequately powered clinical trials with sufficient participants due to rare populations, and
the financial risks of developing drugs for small markets.

Next generation sequencing of the human genome has proven useful for finding pathogenic DNA mutations in rare diseases of childhood. However, many current approaches, such as whole exome sequencing are limited to the approximately 2% of the genome that is protein coding and ignores 98% of the genome that is non-coding. This is significant as the non-coding genome accounts for the majority of trait-associated single-nucleotide polymorphisms in the human genome. Whole genome sequencing can detect genome-wide (coding and non-coding) variations, however each individual has thousands of DNA variants of unknown significance where the vast majority are unlikely to cause disease. Without robust annotation linking these thousands of variants to specific phenotypes, the findings from next generation sequencing studies of individuals are frequently un-interpretable.

Complicating matters is the fact that the genome is epigenetically dynamic, meaning that, during fetal development and into adulthood, there are genomic loci that are uniquely active or inactive at each developmental stage and between specific cell sub-populations. For early life diseases, important pathogenic mutations may be missed by next generation sequencing studies of adult tissues if:
the mutations are located in genomic regions that are inactive in adult tissues, or
the mutations are active only in a small sub-population of cells within a tissue sample composed of numerous other cell populations from diverse lineages, or
the mutations causing disease in fetal, neonatal and pediatric populations are active only at specific developmental stages and those stages are not investigated.

Development stage and focused cell populations are pivotal parameters that must be accounted for when identifying mutations, and this Discovery System incorporates these parameters.

A novel systematic approach, the Discovery System as described herein, is needed to identify critical early life disease-causing genomic mutations and gene expression changes followed by functional validation through robust and repeatable experimentation in model systems of human early life development. The Discovery System provides solutions to the drug development difficulties and to the epigenetically dynamic issues. Further, for large scale production the Discovery System is made automated.

### SUMMARY

Disclosed herein are methods and compositions for identifying disease causing, genomic mutations using high content genomic assays, phenotypic measurements, and Artificial Intelligence/Machine Learning of human induced pluripotent stem cell (hiPSC) disease models. The methods disclosed herein can identify DNA mutations and gene expression changes that cause early life diseases. The methods can focus on key tissues during key developmental epochs to account for intermittent DNA mutation phenotypes.

The genomic mutations and gene expression changes identified and the developmental stage at which the mutants act and expression changes occur can identify targets for drugs and diagnostics. The methods may also identity genes that can be used in gene therapy applications, and/or gene products that can be developed as biologics.

The methods disclosed herein can use stem cells (e.g., hiPSC, adult stem cells, embryonic stem cells, progenitor cells, etc.) which are induced to develop into a target tissue of interest (e.g., cardiac muscle). Genomic mutations and gene expression changes associated with defects in cardiac development can be followed to identify phenotypes that are associated with the genomic mutation and gene expression changes. The methods can also associate the genomic mutation and gene expression changes and their phenotype with a stage of development.

### BRIEF DESCRIPTION OF THE DRAWINGS

FIG. 1 is a schematic of the sourcing of human induced pluripotent stem cells employed in the Discovery System.

FIG. 2 is a flowchart depicting the Discovery System for genomic mutation discovery in human early life disease.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, the terms “amplification” and “amplifying” refer to a polynucleotide amplification reaction, namely, a population of polynucleotides that are replicated from one or more starting sequences. Amplifying may refer to a variety of amplification reactions, including, but not limited to, polymerase chain reaction, linear polymerase reactions, nucleic acid sequence-based amplification, rolling circle amplification and like reactions. Typically, amplification primers are used for amplification, the result of the amplification reaction being an amplicon.

As used herein, the term “benign” means something of little or no effect. For example, genetic variants can be pathogenic or benign. A “benign variant” or “benign genetic variant” is one that has little or no effect in a disease or condition, such as eye or hair color; that is, they are considered part of the normal biology of an individual or organism and thus are often referred to as “normal variants.” Benign variants can also be considered as the opposite of “pathogenic variants,” which are causal of a disease or condition. In some embodiments of the invention, it may be desirable to identify benign variants associated with a particular phenotype that do not cause disease. Such benign variants can be identified with the present invention by use of cohorts affected and unaffected by the phenotype or trait of interest such as a desirable growth characteristic in a plant crop or a particular size or coat color of a companion animal.

The term “detectable phenotype” includes any cellular phenotype that can be detected and used to separate or split one population or pool of cells from another. In particular embodiments, cells of interest can be selected based upon the presence of a detectable phenotype. Examples of detectable phenotypes include, but are not limited to, cell growth, cell survival, reporter gene expression, physical characteristics of the cell (e.g., shape, size, mass, and/or density), cell mobility or migration behavior, cellular appearance or morphology, and combinations thereof. In certain embodiments, a detectable phenotype is used to determine whether a genetic element is phenotypically responsive to a modulating nucleic acid element. In other embodiments, a detectable phenotype is a phenotype that is observed with one (single-mutant phenotype), two (double-mutant phenotype), three, four, five, six, seven, eight, nine, ten, or more mutations and used to identify one or a plurality of genetic elements, one or a plurality of nucleic acid elements that modulate genetic elements, and/or genetic interactions between genetic elements.

As used herein, an “effective amount” or “therapeutically effective amount” are used interchangeably, and defined to be an amount of a compound, formulation, material, or composition, as described herein effective to achieve a particular biological result.

As used herein, the term “expression level” of a gene refers to the amount of RNA transcript that is transcribed by a gene and/or the amount of protein that may be translated from an RNA transcript, e.g. mRNA. For example, for genes which encode a miRNA, the expression level may be determined through quantifying the amount of RNA transcript which is expressed, e.g. using standard methods such as quantitative PCR of a mature miRNA, microarray, or Northern blot. Alternatively, the expression level may also be determined through measuring the effect of a miRNA on a target mRNA.

As used herein, the term “molecular pathway”, also called a biological pathway, is a series of interactions among molecules in a cell that leads to a certain product or a change in a cell. Such a molecular pathway can trigger the assembly of new molecules, such as a fat or protein. Molecular pathways can also turn genes on and off, or spur a cell to move. Importantly, DNA mutations in regulatory regions of the genome can cause changes in molecular pathway activity by inhibiting or activating the expression of key molecules.

As used herein, the term “expression of a gene” or “gene expression” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi).

As used herein, an “expression vector” and an “expression construct” are used interchangeably, and are both defined to be a plasmid, virus, or other nucleic acid designed for protein expression in a cell. The vector or construct is used to introduce a gene into a host cell whereby the vector will interact with polymerases in the cell to express the protein encoded in the vector/construct. The expression vector and/or expression construct may exist in the cell extrachromosomally or integrated into the chromosome. When integrated into the chromosome the nucleic acids comprising the expression vector or expression construct will be an expression vector or expression construct.

As used herein, the term “gene” refers to a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

As used herein, “heterologous” is defined to mean the nucleic acid and/or polypeptide is not homologous to the host cell. Alternatively, “heterologous” means that portions of a nucleic acid or polypeptide that are joined together to make a combination where the portions are from different species, and the combination is not found in nature.

As used herein, the term “next generation sequencing” and/or “high throughput sequencing” and/or “deep sequencing” refer to sequencing technologies having increased throughput as compared to the traditional Sanger- and capillary electrophoresis-based approaches, for example with the ability to generate hundreds of thousands or millions of relatively short sequence reads at a time. Examples of next generation sequencing techniques include, but are not limited to, sequencing by synthesis, sequencing by ligation, and sequencing by hybridization. Examples of next generations sequencing methods include, but are not limited to, pyrosequencing as used by the GS Junior and GS FLX Systems (454 Life Sciences, Bradford, Conn.); sequencing by synthesis as used by Miseq and Solexa system (Illumina, Inc., San Diego, Calif.); the SOLiD™ (Sequencing by Oligonucleotide Ligation and Detection) system and Ion Torrent Sequencing systems such as the Personal Genome Machine or the Proton Sequencer (Thermo Fisher Scientific, Waltham, Mass.), Single Molecule, Real-Time (SMRT) Sequencing (Pacific Biosciences, Menlo Park, Calif.); and nanopore sequencing systems (Oxford Nanopore Technologies, Oxford, united Kingdom).

As used herein, is “normal” refers to a standard or usual state. As applied in biology and medicine, a “normal state” or “normal person” is what is usual or most commonly observed. For example, individuals with disease are not typically considered normal. Example usage of the term includes, but is not limited to, “normal subject,” “normal individual,” “normal organism,” “normal cohort,” “normal group,” and “normal population.” In some cases, the term “apparently healthy” is used to describe a “normal” individual. Thus, an individual that is normal as a child may not be normal as an adult if they later develop, for example, cancer, Alzheimer's disease or are exposed to health-impairing environmental factors such as toxins or radiation. Conversely, a child treated and cured of leukemia can grow up to be an apparently healthy adult. Normal can also be described more broadly as the state not under study. For example, and as used herein, a normal cohort, used in conjunction with a particular disease cohort under investigation, includes individuals without the disease being studied but can also include individuals that have another unrelated disease or condition. Further, a normal group, normal cohort, or normal population can consist of individuals of the same ethnicity or multiple ethnicities, or likewise, same age or multiple ages, all male, all female, male and female, or any number of demographic variables. As used herein, the term “normal” can mean “normal subjects” or “normal individuals.”

As used herein, the term “normal variation” refers to the spectrum of copy number variation, or frequencies of copy number variants, found in a normal cohort or normal population (see “Normal” definition). Normal variation can also refer to the spectrum of variation, or frequencies of variants, found in a normal cohort or normal population for any class of variant found in genomes, such as, but not limited to, single nucleotide variants, insertions, deletions, and inversions.

As used herein, the term “pathogenic” is generally defined as able to cause or produce disease. For example, genetic variants can be pathogenic or benign. In some cases, the term “pathogenic variant” or “pathogenic genetic variant” is more broadly used for a variant associated with or causative of a condition, which may or may or may not be a disease. In some cases, a pathogenic variant can be considered a causative variant or causative mutation, in which case the variant is causal of the disease or condition. Pathogenic variants can also be considered as the opposite of “benign variants,” which are not causal of a disease or condition.

As used herein, the term “reporter” or “reporter molecule” refers to a moiety capable of being detected indirectly or directly. Reporters include, without limitation, a chromophore, a fluorophore, a fluorescent protein, a receptor, a hapten, an enzyme, and a radioisotope.

As used herein, the term “reporter gene” refers to a polynucleotide that encodes a reporter molecule that can be detected, either directly or indirectly. Exemplary reporter genes encode, among others, enzymes, fluorescent proteins, bioluminescent proteins, receptors, antigenic epitopes, and transporters.

As used herein, the term “reporter probe” refers to a molecule that contains a detectable label and is used to detect the presence (e.g., expression) of a reporter molecule. The detectable label on the reporter probe can be any detectable moiety, including, without limitation, an isotope, chromophore, and fluorophore. The reporter probe can be any detectable molecule or composition that binds to or is acted upon by the reporter to permit detection of the reporter molecule.

As used herein, the term “trait”, in the context of biology, refers to a trait that relates to any phenotypical distinctive character of an individual member of an organism, or of an individual cell, in comparison to (any) other individual member of the same organism, or of (any) other individual cell. For example, in the current invention traits (preferably of the same character) of cells (from the same organism) are compared. Within the context of the current invention the trait can be inherited, i.e. be passed along to next generations of the organism by means of the genetic information in the organism. As used herein, the terms “trait of the same character” and “trait of said character” refer to anyone of a group of at least two traits that exist (or became apparent) for a character. For example, in case of the character “color of the flower”, phenotypical manifestations (traits) might comprise blue, red, white, and so on. In the above example blue, red and white are all different traits of the same character.

As used herein, “transfected” or “transformed” or “transduced” are defined to be a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

As used herein, the term “stem cell” is defined as a cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system), but not into extra-embryonic tissues like the placenta. A variety of stem cell types are known in the art and can be used, including for example, embryonic stem cells, adult stem cells, inducible pluripotent stem cells, hematopoietic stem cells, neural stem cells, epidermal neural crest stem cells, mammary stem cells, intestinal stem cells, mesenchymal stem cells, olfactory adult stem cells, testicular cells, and progenitor cells (e.g., neural, angioblast, osteoblast, chondroblast, pancreatic, epidermal, etc.).

### Discovery System

In this Discovery System (300), the thousands of DNA variants identified by whole genome sequencing are winnowed to the true pathogenic mutation(s) through in vitro modeling of fetal, neonatal and pediatric (early life) diseases. The Discovery System (300) employs patient-specific human induced pluripotent stem cell (hiPSC) disease models that mimic the developmental progression of actual tissues.

Because hiPSCs carry the identical genome of the individual from whom they are derived, they also carry the disease-causing DNA mutations that are affecting the individual. Importantly, hiPSC disease models are developmentally immature and fetal-like rather than adult-like. This means that diseases that begin in utero or during childhood are ideally suited for hiPSC disease modeling.

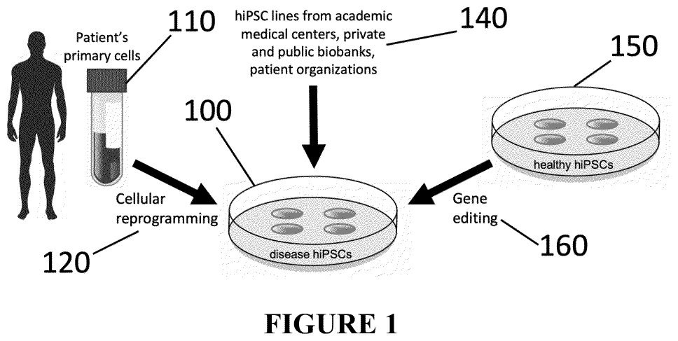

As shown in FIG. 1, the derivation of disease-specific hiPSCs (100) includes:
primary non-pluripotent cells (110), such as fibroblasts or white blood cells, sourced from a patient under study and subjected to conventional methods to reprogram (120) them, including using viral or non-viral reprograming factor methods, to generate hiPSCs;
hiPSC lines from patients acquired from academic medical centers, private and public biobanks, and patient organizations (140); and
hiPSCs derived from healthy individuals (150) that are genetically engineered (160) to carry known or published pathogenic mutations using gene editing methods such as CRISPR/Cas9. In one embodiment, human embryonic stem cells (hESCs) are engineered to carry pathogenic mutations.
   The disease hiPSCs (100) are cultured and expanded using standard human pluripotent cell culture methods. hiPSCs derived from healthy family members or healthy un-related individuals (150), or wildtype human embryonic stem cells, are used in the Discovery System (300) for control comparison evaluation.

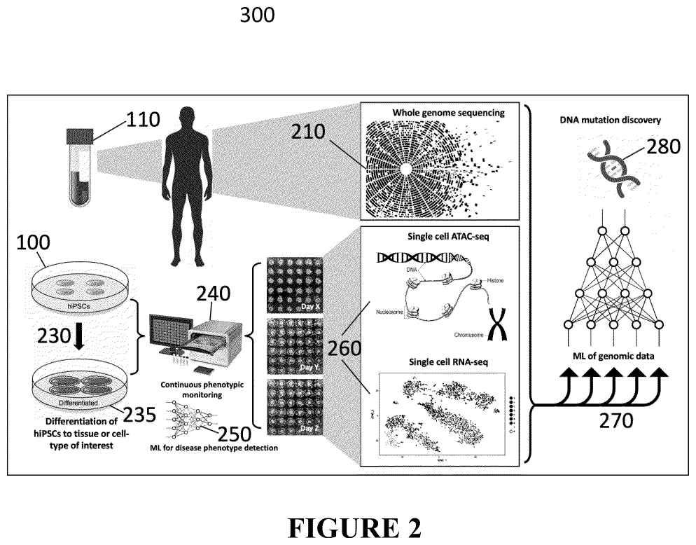

As shown in FIG. 2, the Discovery System (300) combines high content genomic assays, continuous phenotypic monitoring, and Artificial Intelligence/Machine Learning with hiPSC disease models to identify DNA mutations and gene expression changes causing early life diseases. Monitoring for disease in differentiating hiPSCs using high resolution phenotypic detection methods and single cell sequencing technologies identifies the specific cell type and specific timepoint at which a pathogenic DNA mutation or gene expression change causes disease. For example, if a disease phenotype in a sub-population of cells is detected on day 10 of hiPSC differentiation, then those genomic regions that became newly active or inactive in that cell type on day 10 will contain DNA variants with the highest probability of pathogenicity.

Whole genome sequencing (210) is performed on the patients' primary cells (110) to generate a list of potential disease-causing DNA mutations (SNPs, indels, etc.) for each patient. In one embodiment, whole genome sequencing is performed on hiPSCs (100) if an individual's primary cells are unavailable. If specific genetic loci that contain pathogenic “hotspots” are already suspected in a patient, then whole exome sequencing, targeted multi-gene or even single gene sequencing may be performed instead of whole genome sequencing on disease and healthy control samples. In one embodiment, cytogenetic studies are performed in addition to Next Generation Sequencing studies to rule out pathogenic large chromosomal aberrations in each patient.

Disease hiPSCs (100) are differentiated (230) into the cell type or tissue of interest (235) using established protocols. This may include hiPSC-derived 2D or 3D fetal organoids or artificial tissues that contain cells from multiple lineages (e.g. three embryonic germ layer formation (Warmflash, A., Sorre, B., Etoc, F., et al. (2014). A method to recapitulate early embryonic spatial patterning in human embryonic stem cells. Nat Methods 11, 847-854, which is incorporated by reference in its entirety for all purposes) and even vasculature) and are differentiated from hiPSCs using established methods (Warmflash, A., Sorre, B., Etoc, F., et al. (2014). A method to recapitulate early embryonic spatial patterning in human embryonic stem cells. Nat Methods 11, 847-854; Wilson, K. D., Ameen, M., Guo, H., et al. (2020). Endogenous retrovirus-derived lncRNA BANCR promotes cardiomyocyte migration in humans and non-human primates. Dev Cell 54, 694-709. Each is incorporated by reference in its entirety for all purposes). In one embodiment, hiPSCs are differentiated into embryoids that contain all three embryonic germ layers (mesoderm, endoderm, and ectoderm). In another embodiment, standard hiPSC differentiation methods that yield homogenous differentiated cells in monolayers without organoid or embryoid organization are employed.

The Discovery System (300) performs continuous phenotypic monitoring (240) throughout the hiPSC differentiation period in order to determine the timing and degree of disease emergence in hiPSC disease models. In the preferred embodiment, continuous phenotypic monitoring is performed throughout differentiation using fully automated live cell culture instruments that include internal cell culture incubators, internal electrophysiologic sensors, and/or internal microscopes and associated lenses and filters for brightfield, phase, fluorescence and other morphologic and biomarker signal detection. Note that an Incucyte® SX5 Live-Cell Analysis System (Sartorius, Germany) is shown in FIG. 2 as one example of automated phenotypic monitoring. In one embodiment, standard hiPSC culture and differentiation is performed by skilled laboratory personnel without automated instrumentation.

Methods for phenotypic monitoring (240) throughout hiPSC differentiation (230) include live cell microscopy, confocal microscopy, light sheet fluorescence microscopy, biomarker immunostaining and fluorescent microscopy, cell painting (Bray, M. A., Singh, S., Han, H., et al. (2016). Cell Painting, a high-content image-based assay for morphological profiling using multiplexed fluorescent dyes. Nature Protocols 11, 1757-1774, which is incorporated by reference in its entirety for all purposes), flow cytometry, biomarker-targeted fluorescently activated cell sorting (FACS), electrophysiology measurements, sodium, calcium and other electrolyte dynamics, ion channel activity, cellular movement/migration assays, protein/peptide chemistry measurements, intercellular and intracellular signaling, phosphorylation, genetic transcription assays, telomere length, organelle-specific assays such as mitochondrial or endoplasmic reticulum health, cell death or senescence assays, cellular respiration, autophagy, extracellular matrix, immunophenotype, cell or nuclear membrane function, and any other assay(s) needed for assessing cellular function, morphology, health or disease. In one embodiment, hiPSC disease models may be genetically modified prior to differentiation such that a biomarker is inactivated or activated upon disease emergence. This change in biomarker activity is then detected using any of the methods (240) described above.

Artificial Intelligence/Machine Learning (250) applied to these phenotypic monitoring data (240) aids in the identification of the first timepoint at which disease emergence occurs in specific cell sub-populations during differentiation. Artificial Intelligence/Machine Learning applied to microscopy in particular, a form of “computer vision”, is a powerful method for improving the detection of cellular phenotypic differences across multiplexed microscopic images (https://www.nature.com/collections/cfcdjceech (2019). Deep learning in microscopy. Nat Methods, which is incorporated by reference in its entirety for all purposes).

When a disease phenotype is detected in differentiating hiPSCs, then single cell genetic studies (260) are performed immediately before, during, and after the timepoint when disease is first detected. Because hiPSC disease models are composed of mixed cell populations, single cell genetic studies combined with high resolution cellular phenotyping methods (240) identify the specific cell type and associated DNA mutation(s) and/or gene expression change(s) causing a disease phenotype. In the preferred embodiment, both single cell RNA-seq (gene expression) and single cell single cell assay for transposase-accessible chromatin (ATAC)-seq (epigenome “open chromatin”) are employed to determine: (a) disease-specific gene expression in each cellular subtype of a disease model, and (b) open or closed epigenomic regions that are associated with disease emergence. In one embodiment, only single cell RNA-seq is performed. In another embodiment, only ATAC-seq is performed. In another embodiment, any single cell genetic detection method is used to assess single cell genetic activity that may include single cell chromosome conformation capture (e.g. scHi-C), single cell chromatin immunoprecipitation sequencing (scChIP-seq), or any other single cell genetic assay.

DNA variants that occur in or near genomic regions showing dynamic changes (gene expression, open chromatin) at the same time as disease emergence in hiPSC disease models have the highest likelihood of being true pathogenic mutations. DNA mutations such as substitutions, insertions and deletions are those that occur (i) within protein-coding genes (exons or introns) and lead to amino acid changes, or (ii) within non-coding genes such as long non-coding RNAs or microRNAs, or (iii) within regulatory regions such as gene promoters, enhancers, and insulators that induce expression changes in downstream genes (The GTEx Consortium (2020). The GTEx Consortium atlas of genetic regulatory effects across human tissues. Science 369, 1318-1330, which is incorporated by reference in its entirety for all purposes). For comparison, negative control studies of healthy or wildtype hiPSCs are performed using the Discovery System in order to establish negative (healthy) control baseline data. For example, if a gene is highly upregulated in disease hiPSC models but minimally expressed in negative control hiPSC models, then that gene's abberant expression is one potential cause of disease.

By determining both the patient-specific whole genome sequence and genome-wide RNA expression (the “transcriptome”) at specific timepoints during hiPSC differentiation, the Discovery System is also able to detect expressed quantitative trait loci (eQTL). An expression quantitative trait is an amount of an mRNA transcript or a protein that is the product of a single gene with a specific chromosomal location. Chromosomal loci that explain variance in expression traits are called eQTLs. Importantly, the abundance of a gene transcript are directly modified by DNA mutations or polymorphisms in regulatory gene elements such as promoters, enhancers, insulators or untranslated regions (UTRs) (Zhu Z., et al. Integration of summary data from GWAS and eQTL studies predicts complex trait gene targets. Nature Genetics. 2016; 48:481-487, which is incorporated by reference in its entirety for all purposes). Consequently, transcript abundance is a quantitative trait that can be mapped to a specific chromosomal locus with considerable power. The combination of whole genome sequencing and the measurement of global gene expression by RNA-seq allows the systematic identification of eQTLs (The GTEx Consortium (2020). The GTEx Consortium atlas of genetic regulatory effects across human tissues. Science 369, 1318-1330, which is incorporated by reference in its entirety for all purposes). By assaying gene expression and genetic variation simultaneously on a genome-wide basis, statistical methods can be used to map the genetic factors that underpin patient-specific hiPSC disease model differences in quantitative levels of expression of thousands of transcripts.

To confirm DNA variant or gene expression pathogenicity, gene editing methods such as CRISPR are then used to correct these mutations in patient-specific hiPSCs, which are then differentiated to the cell type or tissue of interest with continuous phenotypic monitoring as before. In the case of gene expression changes associated with disease emergence, alteration of the activity or expression of these identified genes in hiPSC disease models followed by repeat disease modeling to determine the degree of disease resolution can be performed with methods that may include RNA interference (RNAi), short hairpin RNA (shRNA), and CRISPR. Amelioration of the disease phenotype in corrected hiPSC models then validate that the candidate DNA mutation or gene expression change is truly pathogenic.

Artificial Intelligence/Machine Learning has transformed the field of genomics and greatly improved the quality and speed of predictions of genetic variation on phenotype (Eraslan, G., Avsec, Z., Gagneur, J., et al. (2019). Deep learning: new computational modelling techniques for genomics. Nat Rev Genet 20, 389-403, which is incorporated by reference in its entirety for all purposes). Artificial Intelligence/Machine Learning analysis (270) of single cell sequencing dynamics around the period of disease emergence in hiPSC disease models narrows the list of DNA variants and gene expression changes to cell type-specific genomic regions whose change in activity correlate with disease emergence. These data can then be used to determine the specific DNA mutation(s) and/or gene expression change(s) that have the highest correlation with disease in a specific cell type (280).

Negative control studies of healthy or wildtype hiPSCs are performed using the Discovery System in order to establish negative (healthy) control baseline data. These control data are also used for ML training sets prior to analysis of disease hiPSC data. In the preferred embodiment, hiPSCs derived from healthy family members of patients are run through the Discovery System. This includes whole genome sequencing of healthy family member primary cell samples (210), hiPSC derivation from healthy family member primary cells (100), differentiation (230, 235), and ML-assisted continuous phenotype monitoring (240, 250). ML-assisted single cell genetic studies (260, 270) are performed on healthy family member hiPSCs at the same timepoint when disease is detected in disease hiPSCs during differentiation. In one embodiment, wildtype hESCs or healthy un-related hiPSCs are used for establishing baseline control studies and ML training sets.

When genetically engineered hiPSCs or hESCs that carry known pathogenic mutations are employed in the Discovery System, then genetic studies (whole genome sequencing, single cell genetic studies) may not be necessary as the pathogenic mutation(s) are already known. However, to rule out off-target DNA mutations that may be a byproduct of the gene editing process, Whole genome sequencing may be used to detect un-intended mutations in engineered cell lines.

Furthermore, important secondary molecular targets may also exist in patients that are directly regulated by a primary DNA mutation. For example, a pathogenic DNA mutation in a regulatory region (e.g. a gene's promoter or enhancer) causes aberrant expression of a nearby gene which then leads to disease. In this scenario, rather than target the primary DNA mutation in the gene's regulatory region, it may be simpler to instead target the secondary aberrantly expressed gene (or its RNA transcript) in order to treat the disease. Therefore, in the preferred embodiment single cell RNA-seq and/or ATAC-seq to discover secondary molecular targets will be performed on gene-edited hiPSCs and hESCs, as well as on hiPSCs from patients with pre-determined primary DNA mutations.

This Discovery System identifies the DNA variants associated with disease emergence in hiPSC disease models and which are the true pathogenic mutations underlying early life disease. Gene editing methods such as CRISPR are then used to remove or correct candidate mutation(s) in patient hiPSC lines, followed by repeat disease modeling to determine the degree of disease resolution.

The Discovery System also identifies cell-type specific gene expression changes associated with disease emergence in hiPSC disease models, and which may also be contributing to early life disease. Alteration of the activity or expression level of these identified genes in hiPSC disease models followed by repeat disease modeling to determine the degree of disease resolution can be performed with methods that may include RNA interference (RNAi), short hairpin RNA (shRNA), and CRISPR.

### Genomic Analysis

“Next generation sequencing” and/or “high throughput sequencing” and/or “deep sequencing” refer to sequencing technologies that have increased throughput as compared to the traditional Sanger- and capillary electrophoresis-based approaches, for example with the ability to generate hundreds of thousands or millions of relatively short sequence reads at a time. Examples of next generation sequencing techniques include, but are not limited to, sequencing by synthesis, sequencing by ligation, and sequencing by hybridization. Examples of next generations sequencing methods include, but are not limited to, pyrosequencing as used by the GS Junior and GS FLX Systems (454 Life Sciences, Bradford, Conn.); sequencing by synthesis as used by Miseq and Solexa system (Illumina, Inc., San Diego, Calif.); the SOLiD™ (Sequencing by Oligonucleotide Ligation and Detection) system and Ion Torrent Sequencing systems such as the Personal Genome Machine or the Proton Sequencer (Thermo Fisher Scientific, Waltham, Mass.); Single Molecule, Real-Time (SMRT) Sequencing (Pacific Biosciences, Menlo Park, Calif.); nanopore sequencing systems (Oxford Nanopore Technologies, Oxford, United Kingdom), and single molecule real time sequencing (Pacific Biosciences, Menlo Park, Calif.).

Whole genome, whole exome, gene panel, and single gene sequencing refer to the region of the genome that is “targeted” by next generation sequencing methods. Whole genome sequencing sequences the entire genome, including both protein coding and noncoding regions. Whole exome sequencing targets only the protein coding genes that comprise the human genome, and does not include noncoding regions other than introns and possibly regions (“untranslated regions”) immediately upstream and downstream of individual genes. Gene panel assays target specific genes that may relate to a disease or biological process, and single gene sequencing targets a specific gene.

RNA-seq refers to the use of next generation sequencing to reveal the presence and quantity of RNA that is expressed genome-wide in a biological sample at a given moment, typically called the “transcriptome”. Because it sequences RNA transcripts, RNA-Seq facilitates detection of alternative gene splicing, post-transcriptional modifications, gene fusions, mutations or SNPs, changes in gene expression over time, and differences in gene expression in different groups or treatments. In addition to messenger RNA (mRNA) transcripts, RNA-Seq can look at different populations of RNA to include total RNA, small RNA, such as microRNA (miRNA), long noncoding RNA (lncRNA), transfer RNA (tRNA), and ribosomal profiling. RNA-Seq can also be used to determine exon/intron boundaries and verify or amend previously annotated 5′ and 3′ gene boundaries. Statistically significant changes in global gene expression between conditions (e.g. different cell types or tissues, disease vs. healthy states, treatment vs. control, etc.) can be determined by comparing the transcriptomes of each condition followed by statistical determination of significantly down or up-regulated genes and groups of genes relative to other conditions. In the case of gene expression, the quantitative unit of measure is typically the “fold change” in gene expression between conditions. To increase statistical confidence, replicates of each sample are subjected to RNA-seq so that there are multiple replicate datasets for each condition. Furtheremore, because the RNA sequence of transcripts are determined by RNA-seq, detected transcripts can be mapped to their corresponding genomic regions from which they are expressed. Doing so enables determination of (i) the specific gene from which the detected RNA is transcribed, and (ii) the nearby regulatory elements (for example, enhancers, promoters, insulators, etc.) that control its expression and may account for differences in gene expression between conditions.

An expression quantitative trait is an amount of an mRNA transcript or a protein. These are usually the product of a single gene with a specific chromosomal location. Chromosomal loci that explain variance in expression traits are called eQTLs. Importantly, the abundance of a gene transcript can be directly modified by DNA mutations or SNPs in regulatory gene elements such as promoters, enhancers, insulators or untranslated regions (UTRs) (Zhu Z., et al. Integration of summary data from GWAS and eQTL studies predicts complex trait gene targets. Nature Genetics. 2016; 48:481-487, which is incorporated by reference in its entirety for all purposes). Consequently, transcript abundance is a quantitative trait that can be mapped with considerable power. The combination of whole-genome genetic association studies and the measurement of global gene expression by RNA-seq allows the systematic identification of eQTLs (The GTEx Consortium (2020). The GTEx Consortium atlas of genetic regulatory effects across human tissues. Science 369, 1318-1330, which is incorporated by reference in its entirety for all purposes). By assaying gene expression and genetic variation simultaneously on a genome-wide basis in a large number of samples, statistical genetic methods can be used to map the genetic factors that underpin individual differences in quantitative levels of expression of many thousands of transcripts. Mapping eQTLs is done using standard QTL mapping methods that test the linkage between variation in expression and genetic polymorphisms. Standard gene mapping software packages can be used, although it is often faster to use custom code such as QTL Reaper or the web-based eQTL mapping system GeneNetwork. GeneNetwork hosts many large eQTL mapping data sets and provide access to fast algorithms to map single loci and epistatic interactions.

Cytogenetics is a clinical and research field of molecular biology that determines the function and overall health of chromosomes and how they affect cell behaviour, particularly to their behaviour during mitosis and meiosis. Techniques used include karyotyping, analysis of G-banded chromosomes, other cytogenetic banding techniques, as well as molecular cytogenetics such as fluorescent in situ hybridization (FISH) and comparative genomic hybridization (CGH).

Single cell genomic technologies utilizes methods and technologies for isolating and sequencing molecules in single cells (Camp J G, et al. Mapping human cell phenotypes to genotypes with single-cell genomics. Science. 2019; 365:1401-1405, which is incorporated by reference in its entirety for all purposes) This technology has become enormously useful in order to understand cellular heterogeneity within biological samples. The most common single cell sequencing application is single cell transcriptomics (whole genome gene expression) in the form of RNA (scRNA-seq). Newer methods allow for assessment of the “accessible” genome such as single cell assay for transposase-accessible chromatin using sequencing (scATAC-seq) (Buenrostro J D, et al. Single-cell chromatin accessibility reveals principles of regulatory variation. Nature. 2015; 523:486-490, which is incorporated by reference in its entirety for all purposes). scATAC-seq determines the regions of the genome that are active and not concealed by regulatory histones and other complexes that comprise chromatin. As this is a rapidly evolving field, numerous new variations of single cell genomics are constantly being introduced.

Camp J G, et al. 2019. In this review, Camp et al. discuss the enduring goal to catalog all human cell types, to understand how they develop, how they vary between individuals, and how they fail in disease. They report that single-cell genomics has revolutionized this endeavor because sequencing-based methods provide a means to quantitatively annotate cell states on the basis of high-information content and high-throughput measurements. Together with advances in stem cell biology and gene editing, scientists are beginning to understand the cellular phenotypes that compose human bodies and how the human genome is used to build and maintain each cell. Camp et al. review recent advances into how single-cell genomics is being used to develop personalized phenotyping strategies that cross subcellular, cellular, and tissue scales to link the human genome to human cumulative cellular phenotypes.

Buenrostro J D, et al. 2015. Buenrostro et al. report that cell-to-cell variation is a universal feature of life that affects a wide range of biological phenomena, from developmental plasticity to tumour heterogeneity. Although recent advances have improved our ability to document cellular phenotypic variation, the fundamental mechanisms that generate variability from identical DNA sequences remain elusive. In their report, they reveal the landscape and principles of mammalian DNA regulatory variation by developing a robust method for mapping the accessible genome of individual cells by assay for transposase-accessible chromatin using sequencing (ATAC-seq) integrated into a programmable microfluidics platform. Single-cell ATAC-seq (scATAC-seq) maps from hundreds of single cells in aggregate closely resemble accessibility profiles from tens of millions of cells and provide insights into cell-to-cell variation. Accessibility variance is systematically associated with specific trans-factors and cis-elements, and they discover combinations of trans-factors associated with either induction or suppression of cell-to-cell variability. They further identify sets of trans-factors associated with cell-type-specific accessibility variance across eight cell types. Targeted perturbations of cell cycle or transcription factor signalling evoke stimulus-specific changes in this observed variability. The pattern of accessibility variation in cis across the genome recapitulates chromosome compartments de novo, linking single-cell accessibility variation to three-dimensional genome organization. Single-cell analysis of DNA accessibility provides new insight into cellular variation of the ‘regulome’.

### Stem Cells and Reprogramming

In cell biology, pluripotency refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system), but not into extra-embryonic tissues like the placenta. In 2006, it was shown that pluripotency can be “induced” in adult or mature cells through introduction of specific embryonic factors (Takahashi K and Yamanaka S. Induction of pluripotent stem cells from mouse embryonic and adult fibroblast cultures by defined factors. Cell. 2006; 126:663-676, which is incorporated by reference in its entirety for all purposes). These factors have become known as the “Yamanaka factors” and are used commonly to “reprogram” (induce pluripotency) in adult cells to transform them to a pluripotent state. The factors used for inducing pluripotency can differ but are generally accepted to include the genes Oct4, Sox2, Klf4 and Myc.

Reprogramming factors can be delivered to adult cells through a variety of techniques (Abbar, A. A., Ngai, S. C., Nograles, N., et al. (2020). Induced pluripotent stem cells: Reprogramming platforms and applications in cell replacement therapy. BioResearch Open Access 9, 121-136. Liu, G., David, B. T., Trawczynski, M., et al. (2020). Advances in pluripotent stem cells: History, mechanisms, technologies, and applications. Stem Cell Reviews and Reports 16, 3-32. Teshigawara, R., Cho, J., Kameda, M., et al. (2017). Mechanism of human somatic reprogramming to iPS cell. Laboratory Investigation 97, 1152-1157. Each is incorporated by reference in its entirety for all purposes) including non-integrating virus delivery (e.g. Sendai virus), genome-integrating retrovirus or lentivirus delivery, non-viral episomal gene vectors, minicircles, and non-viral delivery of synthetic RNA, small molecules, microRNAs, mRNA or proteins. Examples of commercial kits for inducing pluripotency in adult cells include CytoTune-iPSC 2.0 Sendai Reprogramming Kits (ThermoFisher Scientific, Waltham, MA), QualiStem Episomal iPSC Repgoramming Kit (Creative Bioarray, Shirley, NY), and Human iPS Cell Reprogramming Episomal Kit (ALSTEM Cell Advancements, Richmond, CA), Episomal Repgoramming System (System Biosciences, Palo Alto, CA), The adult cells used for induction of pluripotency may include primary non-pluripotent cells, such as fibroblasts or white blood cells, sourced from a patient under study. Once pluripotency is induced and hiPSC colonies are manually picked and transferred to a new culture apparatus, hiPSCs can then be maintained in culture media such as mTeSR (Stem Cell Technologies, Vancouver, Canada) and Essential 8 (ThermoFisher Scientific), among others.

The known phenotypic variability seen across different hiPSC lines, as well as the time- and resource-intensive nature of hiPSC reprogramming, can be optimized by implementing automated solutions for cell reprogramming and hiPSC expansion. This includes automated, modular platforms covering the entire process of hiPSC production, ranging from adult human primary cell expansion, Sendai virus-based reprogramming to automated isolation, and parallel expansion of hiPSC clones (Elanzew, A., NieBing, B., Langendoerfer, D., et al. (2020). The StemCellFactory: A modular system integration for automated generation and expansion of human induced pluripotent stem cells. Front Bioeng Biotechnol 8, 580352, which is incorporated by reference in its entirety for all purposes). Robotic liquid handling units that deliver footprint-free hiPSCs can be achieved and with high efficiency. Evolving hiPSC colonies are automatically detected, harvested, and clonally propagated. To ensure high fidelity performance, a high-speed microscope may be implemented for in-process quality control, and image-based confluence measurements for automated dilution ratio calculation. Such a set-up will enable automated, user-independent expansion of hiPSCs under fully defined conditions, and can generate a large number of hiPSC lines for disease modeling, and drug screening at industrial scale, and quality.

### Cellular Differentiation

hiPSCs (100) can be differentiated into the cell type or tissue of interest using established protocols (see Indications section below for a comprehensive list of cell types with published protocols). Examples of differentiation protocols include:

Cardiomyocytes. An example protocol for cardiomyocyte differentiation (Burridge, P. W., Matsa, E., Shukla, P., et al. (2014). Chemically defined generation of human cardiomyocytes. Nat Methods 11, 855-860, which is incorporated by reference in its entirety for all purposes). Chemically defined generation of human cardiomyocytes. Nat Methods 11, 855-860, which is incorporated by reference in its entirety for all purposes) is as follows. Briefly, differentiation medium consisting of RPMI-1640 media (Life Technologies) supplemented with B27@ minus insulin (Life Technologies) (RPMI+B27 minus) is used. To this medium, various small molecules are added over a week-long timetable as previously described (Burridge, P. W., Matsa, E., Shukla, P., et al. (2014). Chemically defined generation of human cardiomyocytes. Nat Methods 11, 855-860, which is incorporated by reference in its entirety for all purposes). On the first day (D0) of hiPSC differentiation, 6 μM CHIR 99021 (LC Laboratories) is added. On D2, the medium is aspirated and replaced with RPMI+B27 minus. On D3, the medium is aspirated and replaced with 5 μM of IWR-1 (Selleck Chemicals) in RPMI+B27 minus. The medium is replaced with RPMI+B27 minus on D5 and RPMI plus B27 supplemented with insulin (Life Technologies) (RPMI+B27) on D7. Cardiomyocytes can be maintained in RPMI+B27 with media change every other day. Cardiomyocytes generally begin spontaneously beating between D7-D10. A glucose starvation step further purifies cardiomyocyte culture if needed.

Skeletal muscle. An example protocol for skeletal muscle differentiation (van der Wal, E., Bergsma, A. J., van Gestel, T. J. M. et al. (2017). GAA deficiency in Pompe Disease is alleviated by exon inclusion in hiPSC-derived skeletal muscle cells. Molecular Therapy: Nucleic Acis 7, 101-115, which is incorporated by reference in its entirety for all purposes). Briefly, 0.6 mm large hiPSC colonies cultured in 10 cm dishes on MEF feeders are treated for 5 days with 3.5 mM CHIR99021 (Axon Medchem) in myogenic differentiation medium (DMEM/F12, ITS-X and Penicillin/Streptomycin/Glutamine, all Gibco). CHIR99021 is removed and cells are cultured in myogenic differentiation medium containing 20 ng/mL FGF2 (Prepotech) for 14 days and then cultured for an additional 16 days in myogenic differentiation medium only. Medium is refreshed daily. Purification of Myogenic Progenitors: Using FACS following the 35-day protocol for differentiating hiPSCs into a mixture of cells including myogenic progenitors, cells were harvested and purified by FACS. To this end, cells are washed with PBS, incubated for 5 min with TrypLe (Gibco) at 37 C, and gently detached with a pipetboy. The cell suspension is filtered through a 40 mM FACS strainer (Falcon) to remove cell aggregates. Cells are centrifuged for 4 min at 1,000 rpm and incubated with anti-HNK-1-FITC (1:100, Aviva Systems Biology) and anti-C-MET-APC (1:50, R&D Systems) antibodies for 30 min on ice in myogenic differentiation medium. Cells are washed three times with ice-cold 1% BSA in PBS before FACS sorting. Hoechst (33258, Life Technologies) was used as viability marker. Hoechst/C-MET-positive cells are sorted with a 100 mm nozzle and collected in ice-cold hiPSC-myogenic progenitor proliferation medium (iPSC-MPC-pro medium) containing DMEM high glucose (Gibco) supplemented with 100 U/mL Penicillin/Streptomycin/Glutamine (LifeTechnologies), 10% fetal bovine serum (Hyclone, Thermo Scientific), and 100 ng/mL FGF2 (PeproTech). To reduce cell death, medium is supplemented with RevitaCell Supplement (Gibco) during collection and the first 24 hr of cell culture. Sorting time is limited to 20 min per well. Plates/well are coated for 30 min at room temperature with ECM (E6909-5 mL, 1:200 in iPSC-MPC-pro medium, Sigma Aldrich). Sorted cells are plated either at 40,000 cells in one well of a 48-well plate or at 80,000 cells in one well of a 24-well plate, depending on the amount of cells. Expansion of Myogenic Progenitors: At 1 day after plating FACS sorted myogenic progenitors, the medium is refreshed with iPSC-MPC-pro medium. When cells reach 90% confluence, cells are passaged using diluted TrypLein PBS and plated on ECM-coated plastic. Differentiation of Myogenic Progenitors into Multinucleated Myotubes: For differentiation to multinucleated myotubes, myogenic progenitors are grown to 90% confluence, and the medium is then replaced with myogenic differentiation medium (DMEM/F12, ITS-X and penicillin/streptomycin/glutamine, all Gibco). After 4 days, myotubes are harvested

Motor neurons. An example protocol for motor neuron differentiation (Bianchi, F., Malboubi, M., Li, Y., et al. (2018). Rapid and efficient differentiation of functional motor neurons from human iPSC for neural injury modelling. Stem Cell Research 32, 126-134, which is incorporated by reference in its entirety for all purposes). Briefly, confluent hiPSCs are dissociated using Accutase and plated on tissue culture plates in neural induction medium (NIM), consisting of a 1:1 mix of KO-DMEM/F:12 and neurobasal medium (NBM) supplemented with 10% KnockOut Serum Replacement, 1% Non-Essential Amino Acids (NEAA), 1% GlutaMAX, 0.1 mM 1-ascorbic acid (L-AA, Sigma-Aldrich), 2 μM SB431542 (Cell Guidance Systems), 3 μM CHIR99021 (Sigma Aldrich), 1 μM dorsomorphin (StemCell) and 1 μM compound E (StemCell). 1% RevitaCell was added for the first 24 h only. NIM is replaced daily for six days, after which cells are dissociated with Accutase, and plated in neural progenitor cell (NPC) expansion medium, consisting of a 1:1 mix of KO-DMEM:F12 and NBM, supplemented with 10% P/S, 10% B27, 10% N2, 10% NEAA, 10% GlutaMAX, 0.1 mM L-AA, 10 ng/mL bFGF and 10 ng/mL EGF. NPCs are then cultured for 6 days in motor neuron (MN) induction medium, consisting of a 1:1 mix of KO-DMEM:F12 and Neurobasal Medium supplemented with 1% P/S, 10% B27, 10% N2, 10% Non-Essential Amino Acids, 10% GlutaMAX, 0.1 mM 1-ascorbic acid, 10 μM all-trans retinoic acid (Sigma Aldrich), 100 ng/ml recombinant SHH, 1 μM Purmorphamine (Abcam) and 1 mM SAG Dihydrochloride (Sigma Aldrich). After seven days, cells are dissociated using Accutase, and re-plated in maturation medium, consisting of 1:1 KO-DMEM:F12 and NBM, supplemented with 10% P/S, 10% B27, 10% N2, 10% NEAA, 10% GlutaMAX, 0.1 mM L-AA, 10 ng/mL CNTF, 10 ng/ml BDNF, 10 ng/mL NT-3 and 10 ng/mL GDNF.

Midbrain dopamine neurons. An example protocol for midbrain dopamine neuron differentiation (Tomishima, M. (2012). Midbrain dopamine neurons from hESCs. StemBook, ed. The Stem Cell Research Community, StemBook, doi/10.3824/stembook.1.70.1, https://www.stembook.org, which is incorporated by reference in its entirety for all purposes). Accutase treat hiPSCs for 30-45 minutes, until all colonies are single cells. Pipet Accutase into 15 ml conical with hiPSC media. at least two volumes of hESC to one volume of Accutase. Centrifuge for 5 minutes at 200×g, room temperature. Gelatin treat a new tissue culture dish during the centrifugation. Resuspend cells in hiPSC media with 10 μM Y-27632. Aspirate gelatin from culture dish. Add hiPSCs to gelatinized dish for 1 hour at 37° C. in the incubator. While incubating, prepare a Matrigel-coated plate (1:20 in DMEM or hESC media). After the hour, collect the non-adherent cells from the incubator and gently wash the dish. Centrifuge cells as above. Count cells and plate on Matrigel-treated dishes in hiPSC media with 10 ng/ml FGF2 and 10 μM Y-27632. Plate at 200,000 cells/cm2. At this density, cells should be confluent overnight. If they are not confluent, continue expansion until they are and then induce differentiation. Begin differentiation: Day 0—initiation. Aspirate hiPSC media and add SRM with 100 nM LDN193189/10 μM SB431542. Day 1—SRM/LDN/SB with 100 ng/ml SHH and 2 μM Purmorphamine. Day 2—SRM/LDN/SB/SHH/Purm. Day 3—SRM/LDN/SB/SHH/Purm/3 μM CHIR 99021. Day 4—no feed. Day 5-75% SRM/25% N2 with LDN/SHH/Purm/CHIR. Day 6—no feed. Day 7-50% SRM/50% N2 with LDN/CHIR. Day 8—no feed. Day 9-25% SRM/75% N2 with LDN/CHIR. Day 10—no feed. Day 11—NeuroBasal/B27 with CHIR/BDNF/AA/GDNF/cAMP/TGFB3/10 μM DAPT (put poly-L-ornithine solution on plate overnight in incubator). Day 12—no feed (aspirate poly-ornithine, wash 3 times with PBS, and add fibronectin/laminin overnight in incubator). Day 13—Passage 1:1 onto poly-L-ornithine/fibronectin/laminin-coated dishes with 30-45 minutes of Accutase treatment. Spin down in NB/B27, and resuspend in NB/B27 with BAGCT and DAPT (same as above without CHIR). Day 14—no feed. Day 15—from here, keep the same media composition and feed every other day. Between D20-25 when cells become bipolar and make space on the dish, passage them again to poly-L-ornithine/fibronectin/laminin-coated dishes using Accutase. Replate 300-400K per 24 well/well or 2-3 million per 6 well/well.

Hepatocytes. An example protocol for hepatocyte differentiation (Gieseck, R. L., Hannan, N. R. F., Bort, R., et al. (2014). Maturation of induced pluripotent stem cell derived hepatocytes by 3D-culture. PloS One 9, e86372, which is incorporated by reference in its entirety for all purposes). Maturation of induced pluripotent stem cell derived hepatocytes by 3D-culture. PloS One 9, e86372, which is incorporated by reference in its entirety for all purposes) is as follows. hiPSC lines are split (day 0) and maintained for 48 hrs in CDM-PVA supplemented with Activin A and FGF2 (media is changed daily for all subsequent steps, and cells are differentiated at 37° C., 5% CO2, 5% O2, unless stated otherwise). On days 2-3, cells are differentiated in CDM-PVA supplemented with Activin A (100 ng/mL), FGF2 (80 ng/mL), BMP4 (10 ng/mL; R&D), 10 μM LY-294002 (Promega), and 3 μM Stemolecule CHIR99021 (StemGent). On day 4, cells are differentiated in CDM-PVA supplemented with Activin A (100 ng/mL), FGF2 (80 ng/mL), BMP4 (10 ng/mL; R&D), and 10 μM LY-294002. On day 5, cells are differentiated in RPMI Medium (RPMI 1640 Medium, GlutaMAX (Invitrogen), 2% B-27 Serum-Free Supplement (50×) (Invitrogen), 1% MEM Non-Essential Amino Acids Solution (100×) (Invitrogen), 1% penicillin/streptomycin) supplemented with Activin A (100 ng/mL) and FGF2 (80 ng/mL). On day 6, cells are expanded in RPMI medium supplemented with Activin A (50 ng/mL). On day 7, cells are split using Cell Dissociation Buffer (Enzyme-free, Hank's; Invitrogen) and plated in gelatin-coated, MEF media conditioned 6-well plates at a density of 105,000 cells/cm2 in RPMI+Activin A (50 ng/mL)+Y-27632 2HCl (10 μM Selleck chem). Cells are maintained in RPMI+Activin A (50 ng/mL) on days 8-9. From day 10 onward, cells are matured in Hepatozyme-SFM (Invitrogen) supplemented with 1% 200 mM L-glutamine, 1% penicillin/streptomycin, 2% MEM Non-Essential Amino Acids Solution (100×), 2% chemically defined lipid concentrate, 0.14% insulin, 0.28% transferrin, hepatocyte growth factor (50 ng/mL, Peprotech), and oncostatin M (10 ng/mL, R&D) with media changed every other day.

Pancreatic differentiation. An example protocol for pancreatic differentiation (Nostro, M. C., Sarangi F., Ogawa, S., Holtzinger, et al. (2012). Pancreatic differentiation. StemBook, ed. The Stem Cell Research Community, StemBook, doi/10.3824/stembook.1.72.1, https://www.stembook.org, which is incorporated by reference in its entirety for all purposes). Day 0: Stage 1 Endoderm Progenitors. Remove the medium from hiPSCs and wash once with RPMI. To each well, add 1 mL of media containing ActA, WNT3a. Incubate for 24 hours at 37° C. in a 5% CO2 incubator. Day 1-2: Stage 1 Endoderm Progenitors. There will be some debris in the cultures after 24 hours. Remove media and wash once with RPMI. To each well, add 1 mL of media containing Ascorbic acid, BMP4, bFGF, ActA, VEGF. Incubate for 24 hours at 37° C. in a 5% CO2 incubator. Repeat steps 1-2 at day 2. Note: Endoderm induction should be evaluated by flow cytometric analysis, monitoring the cells for expression of CXCR4 (CD184) and CD117 (c-KIT). As each hiPSC line has its own unique kinetics, it is best to define the endoderm stage based on the CXCR4/CD117 profile rather than by time in culture. The endoderm stage is defined by the appearance of a population that co-expresses CXCR4 and CD117. Day 3: Harvest for Flow Cytometry. Aspirate the medium and add 1 mL of TRYPSIN-EDTA. Incubate in a 37° C. incubator for 2-3 minutes and then stop the reaction with 1 mL of STOP MEDIUM+DNase. Spin for 5 min at 1000 RPM, aspirate and resuspend in PBS (—Ca2+Mg2+)+10% FCS (usually 500 uL per well harvested). Pass the cells through a 70 um filter to remove any clumps that are still remaining. Stain with the desired antibodies (CXCR4, CD117) according to product datasheets and perform flow cytometric analysis. Day 3, 5: Stage 2 Foregut/Midgut Endoderm. There will be some debris in the cultures after 24 hours. Remove media and wash once with RPMI. To each well, add 1 mL of media containing FGF10, WNT. Incubate for 48 hours at 37° C. in a 5% CO2 incubator. On day 5, remove media. To each well, add 1 mL of media containing FGF10, WNT. Incubate for 24 hours at 37° C. in a 5% CO2 incubator. Day 6-8: Stage 3 Pancreatic Endoderm. Remove media. To each well, add 1 mL of media containing B27, Ascorbic acid, Cyclopamine, retinoic acid (RA), Noggin, FGF10. Incubate for 24 hours at 37° C. in a 5% CO2 incubator. Repeat steps 1-2 on day 7 and 8. Day 9, 11: Stage 4 Endocrine Progenitors. Remove media. To each well, add 1 mL of media containing B27, Ascorbic acid, SB431542, Noggin. Incubate for 48 hours at 37° C. in a 5% CO2 incubator. On day 11, remove media. To each well, add 1 mL of media Incubate for 48 hours at 37° C. in a 5% CO2 incubator. Day 13-20: Stage 5 Endocrine Cells. Remove media. To each well, add 1 mL of media containing Ascorbic acid, SB431542, Noggin, Gamma Secretase Inhibitor. Incubate for 72 hours at 37° C. in a 5% CO2 incubator. Feed every three days. During the course of this time hormone-expressing cells aggregate with each other and form clusters visible by eye. Harvest at day 20. Note: The percentage of endocrine cells should be evaluated by flow cytometric analysis, monitoring the cells for expression of C-Peptide and GCG.

Lung epithelium. An example protocol for lung epithelium differentiation (Jacob, A., Morley, M., Hawkins, F. et al. (2017). Differentiation of human pluripotent stem cells into functional lung alveolar epithelial cells. Cell Stem Cell, 21 472-488, which is incorporated by reference in its entirety for all purposes). Directed Differentiation of hiPSCs into NKX2-1+lung progenitors: Briefly, cells maintained in mTESR1 media are differentiated into definitive endoderm using the STEMdiff Definitive Endoderm Kit (StemCell Technologies), with 1 day addition of supplement A and B, and 2 days addition of supplements B only (Day 4 in the STEMdiff kit protocol). After the endoderm-induction stage, cells are dissociated for 1-2 min at room temperature with GCDR and passaged at a ratio between 1:2 to 1:6 into 6 well plates pre-coated with growth factor reduced matrigel in “DS/SB” anteriorization media, consisting of complete serum-free differentiation medium (cSFDM) base, including IMDM (ThermoFisher) and Ham's F12 (ThermoFisher) with B27 Supplement with retinoic acid (Invitrogen, Waltham, MA), N2 Supplement (Invitrogen), 0.10% bovine serum albumin Fraction V (Invitrogen), monothioglycerol (Sigma), Glutamax (ThermoFisher), ascorbic acid (Sigma), and primocin with supplements of 10 μm SB431542 (“SB”; Tocris) and 2 μm Dorsomorphin (“DS”; Stemgent). For the first 24 hr after passaging, 10 μm Y-27632 is added to the media. After anteriorization in DS/SB media for 3 days (72 hr), cells are cultured in “CBRa” lung progenitor-induction media for 9-11 days. “CBRa” media consists of cSFDM containing 3 μm CHIR99021 (Tocris), 10 ng/mL recombinant human BMP4 (rhBMP4, R&D Systems), and 100 nM retinoic acid (RA, Sigma). On Day 15 of differentiation, efficiency of specification of NKX2-1+lung progenitors is evaluated either by flow cytometry for intracellular NKX2-1 protein, NKX2-1GFP reporter expression, or by expression of surrogate cell surface markers CD47hi/CD26lo. Cell sorting of NKX2-1+Lung Progenitors: On day 15 of differentiation, cells are incubated at 37° C. in 0.05% trypsin-EDTA (Invitrogen) for 7-15 min, until they reach single cell suspension. Cells are then washed in media containing 10% fetal bovine serum (FBS, ThermoFisher), centrifuged at 300 g×5 min, and resuspended in sort buffer containing Hank's Balanced Salt Solution (ThermoFisher), 2% FBS, 10 μm Y-27632, and 10 μm calcein blue AM (Life Technologies) for dead cell exclusion. Cells not containing the NKX2-1GFP reporter are subsequently stained with CD47-PerCPCy5.5 and CD26-PE antibodies (mouse monoclonal; Biolegend 1:200; 1×106 cells in 100 μl) for 30 min at 4° C., washed with PBS, and resuspended in sort buffer. Cells are passed through a 40 μm strainer prior to sorting (Falcon). Various live cell populations indicated in the text (i.e., GFP+, GFP−, CD47hi/CD26−, CD47lo) are sorted on a high-speed cell sorter (MoFlo Legacy). NKX2-1+Lung Progenitor Outgrowth into Alveolar Epithelial Cells: Day 15 cells, either sorted (as described above) or unsorted (dissociated as described above without sorting), are resuspended in undiluted growth factor-reduced matrigel (Corning) at a dilution of 25-100 cells/μl, with droplets ranging in size from 20 μL in 96 well plates to 1 ml in 10 cm tissue culture-treated dishes (Corning). Cells in 3D matrigel suspension are incubated at 37° C. for 20-30 min, then warm media is added to the plates. Outgrowth and distal/alveolar differentiation of cells after day 15 is performed in “CK+DCI” medium, consisting of cSFDM base, with 3 μm CHIR99021, 10 ng/mL rhKGF, and 50 nM dexamethasone (Sigma), 0.1 mM 8-Bromoadenosine 3′,5′-cyclic monophosphate sodium salt (Sigma) and 0.1 mM 3-Isobutyl-1-methylxanthine (IBMX; Sigma) (DCI). Immediately after replating cells on Day 15 10 μm Y-27632 is added to the medium for 24 hr. Additional growth factors or cytokines were added, including FGF10, TGFb, EGF, OSM, TNFα, and IL-1β.

### Organoid Differentiation

Standard hiPSC differentiation methods often yield homogenous differentiated cells in monolayers or sheets without multilineage organoid or embryoid organization. Organoids are more complex than homogenous cell cultures, and can better mimic the biology of human tissues and organs (Kim, J., Koo, B.-K., and Knoblich, J. A. (2020). Human organoids: model systems for human biology and medicine. Nat Rev Mol Cell Biol 21, 571-584, which is incorporated by reference in its entirety for all purposes). Such organoid differentiation methods may include hiPSC-derived 2D or 3D fetal discoids, spheroids, organoids, and engineered artificial tissues that contain cells from multiple lineages (e.g. three embryonic germ layer formation (Warmflash, A., Sorre, B., Etoc, F., et al. (2014). A method to recapitulate early embryonic spatial patterning in human embryonic stem cells. Nat Methods 11, 847-854, which is incorporated by reference in its entirety for all purposes) and even vasculature) and are differentiated from hiPSCs using established methods (Warmflash, A., Sorre, B., Etoc, F., et al. (2014). A method to recapitulate early embryonic spatial patterning in human embryonic stem cells. Nat Methods 11, 847-854; Wilson, K. D., Ameen, M., Guo, H., et al. (2020). Endogenous retrovirus-derived lncRNA BANCR promotes cardiomyocyte migration in humans and non-human primates. Dev Cell 54, 694-709. Each is incorporated by reference in its entirety for all purposes). hiPSCs can also be differentiated into embryoids that contain all three embryonic germ layers (mesoderm, endoderm, and ectoderm) that mimic development of a human embryo in utero.

Kim, J., Koo, B.-K., and Knoblich, J. A. (2020). In their review, Kim et al. argue that historical reliance of biological research on the use of animal models has sometimes made it challenging to address questions that are specific to the understanding of human biology and disease. But with the advent of human organoids—which are stem cell-derived 3D culture systems—it is now possible to re-create the architecture and physiology of human organs in remarkable detail. Human organoids provide unique opportunities for the study of human disease and complement animal models. Human organoids have been used to study infectious diseases, genetic disorders and cancers through the genetic engineering of human stem cells, as well as directly when organoids are generated from patient biopsy samples. Kim et al. review the applications, advantages and disadvantages of human organoids as models of development and disease and outlines the challenges that have to be overcome for organoids to be able to substantially reduce the need for animal experiments.

Warmflash, A., Sorre, B., Etoc, F., et al. (2014). A method to recapitulate early embryonic spatial patterning in human embryonic stem cells. Nat Methods 11, 847-854. Warmflash et al. show that geometric confinement is sufficient to trigger self-organized patterning in hESCs. In response to BMP4, colonies reproducibly differentiated to an outer trophectoderm-like ring, an inner ectodermal circle and a ring of mesendoderm expressing primitive-streak markers in between. Fates were defined relative to the boundary with a fixed length scale: small colonies corresponded to the outer layers of larger ones. Inhibitory signals limited the range of BMP4 signaling to the colony edge and induced a gradient of Activin-Nodal signaling that patterned mesendodermal fates. These results demonstrate that the intrinsic tendency of stem cells to make patterns can be harnessed by controlling colony geometries and provide a quantitative assay for studying paracrine signaling in early development. Importantly, hiPSCs can also be employed instead of hESCs in these published methods.

Wilson, K. D., Ameen, M., Guo, H., et al. (2020). Endogenous retrovirus-derived lncRNA BANCR promotes cardiomyocyte migration in humans and non-human primates. Dev Cell 54, 694-709. Wilson et al. use primate hESC and hiPSC-derived cardiomyocytes that mimic fetal cardiomyocytes in vitro to discover hundreds of novel mRNA transcripts from the primate-specific MER41 family, some of which are regulated by the cardiogenic transcription factor TBX5. The most significant of these are located within BANCR, a long non-coding RNA (lncRNA) exclusively expressed in primate fetal cardiomyocytes. Functional studies using geometrically-patterned hiPSC and hESC-derived cardiac organoids (“cardioids”) revealed that BANCR promotes cardiomyocyte migration in vitro and ventricular enlargement in vivo. They conclude that recently evolved TE loci such as BANCR may represent potent de novo developmental regulatory elements that can be interrogated with species-matching pluripotent stem cell models.

One example of a protocol for creating 2D cardiac organoids involves seeding single cell suspensions of hiPSC lines into stencils (circular stencils with holes for patterning single or arrayed colonies in each well of a tissue culture plate) prior to cardiac differentiation, as described (Myers, F. B., Silver, J. S., Zhuge, Y., et al. (2013). Robust pluripotent stem cell expansion and cardiomyocyte differentiation via geometric patterning. Integr Biol 5, 1495-1506, which is incorporated by reference in its entirety for all purposes). hiPSCs are then incubated on stencils in 37° C. for a minimum of 1 hr to allow cells to settle onto a previously deposited Matrigel matrix within stencil holes. After this time, E8 medium+10 uM ROCK Inhibitor (Sigma Aldrich) are added per well and stencils then carefully removed with forceps, leaving a single colony or arrayed colonies in each well depending on the configuration of the stencils. Media is changed the following day and cells are allowed to fill in each stencil over the following two days. The confluence of the cells are carefully tracked to ensure that cells reached 95-100% confluence at the start of differentiation. Cardiac differentiation is then initiated as described earlier.

Myers, F. B., Silver, J. S., Zhuge, Y., et al. 2013. Myers et al. report that geometric factors including the size, shape, density, and spacing of pluripotent stem cell colonies play a significant role in the maintenance of pluripotency and in cell fate determination. These factors are impossible to control using standard tissue culture methods. As such, there can be substantial batch-to-batch variability in cell line maintenance and differentiation yield. The authors demonstrate a simple, robust technique for pluripotent stem cell expansion and cardiomyocyte differentiation by patterning cell colonies with a silicone stencil. They observed that patterning hiPSC colonies improves the uniformity and repeatability of their size, density, and shape. Uniformity of colony geometry leads to improved homogeneity in the expression of pluripotency markers SSEA4 and Nanog as compared with conventional clump passaging. Patterned cell colonies are capable of undergoing directed differentiation into spontaneously beating cardiomyocyte clusters with improved yield and repeatability over unpatterned cultures seeded either as cell clumps or uniform single cell suspensions. Circular patterns resulted in a highly repeatable 3D ring-shaped band of cardiomyocytes which electrically couple and lead to propagating contraction waves around the ring. Because of these advantages, the authors argue that geometrically patterning stem cells using stencils offer greater repeatability from batch-to-batch and person-to-person, an increase in differentiation yield, a faster experimental workflow, and a simpler protocol to communicate and follow. Furthermore, the ability to control where cardiomyocytes arise across a culture well during differentiation will greatly aid the design of electrophysiological assays for drug-screening.

### Phenotype Monitoring

Methods for phenotypic monitoring (240) throughout hiPSC differentiation (230) include live cell microscopy, confocal microscopy, light sheet fluorescent microscopy, biomarker immunostaining and fluorescent microscopy, cell painting (Bray, M. A., Singh, S., Han, H., et al. (2016). Cell Painting, a high-content image-based assay for morphological profiling using multiplexed fluorescent dyes. Nature Protocols 11, 1757-1774, which is incorporated by reference in its entirety for all purposes), flow cytometry, biomarker-targeted fluorescently activated cell sorting (FACS), electrophysiology measurements, sodium, calcium and other electrolyte dynamics, ion channel activity, cellular movement/migration assays, protein/peptide chemistry measurements, intercellular and intracellular signaling, phosphorylation, genetic transcription assays, telomere length, organelle-specific assays such as mitochondrial or endoplasmic reticulum health, cell death or senescence assays, cellular respiration, autophagy, extracellular matrix, immunophenotype, cell or nuclear membrane function, and any other assay(s) needed for assessing cellular function, morphology, health or disease. In one embodiment, hiPSC disease models may be genetically modified prior to differentiation such that a biomarker is inactivated or activated upon disease emergence. This change in biomarker activity is then detected using any of the methods (240) described above.

### Artificial Intelligence/Machine Learning (Deep Learning)

Artificial Intelligence/Machine Learning (250) applied to phenotypic monitoring data (240) aids in the identification of the first timepoint at which disease emergence occurs in specific cell sub-populations during differentiation. Artificial Intelligence/Machine Learning applied to microscopy in particular, a form of “computer vision”, is a powerful method for improving the detection of cellular phenotypic differences across multiplexed microscopic images (Deep learning in microscopy (https://www.nature.com/collections/cfcdjceech). Nat Methods. 2019, which is incorporated by reference in its entirety for all purposes). Artificial Intelligence/Machine Learning has also transformed the field of genomics and greatly improved the quality and speed of predictions of genetic variation on phenotype (Eraslan G, et al. Deep learning: new computational modelling techniques for genomics. Nat Rev Genet. 2019; 20:389-403, which is incorporated by reference in its entirety for all purposes). Artificial Intelligence/Machine Learning analysis (270) of single cell sequencing dynamics around the period of disease emergence in hiPSC disease models narrows the list of DNA variants to cell type-specific genomic regions whose change in activity correlate with disease emergence. These data can then be used to determine the specific DNA mutation(s) that have the highest correlation with disease in a specific cell type (280).

Eraslan G, et al. 2019. In this review, Eraslan et al. discuss ho, genomics, data-driven science, largely utilizes machine learning to capture dependencies in data and derive novel biological hypotheses. However, the ability to extract new insights from the exponentially increasing volume of genomics data requires more expressive machine learning models. By effectively leveraging large data sets, deep learning has transformed fields such as computer vision and natural language processing. Now, it is becoming the method of choice for many genomics modelling tasks, including predicting the impact of genetic variation on gene regulatory mechanisms such as DNA accessibility and splicing.

Deep learning in microscopy (https://www.nature.com/collections/cfcdjceech). Nat Methods. 2019. In the December 2019 issue of the journal Nature Methods, a collection of articles are featured here that focus on Deep Learning in Microscopy. Examples of the topics covered by this web collection of original research articles and reviews are Deep Learning in imaging, Deep learning advances in super-resolution imaging, Deep learning for cellular image analysis, applications of Deep learning for fluorescence image reconstruction, and more.

Temporal Detection of Disease in hiPSC Models Using Deep Learning

A key innovation of The Discovery System is continuous phenotypic monitoring throughout the hiPSC differentiation period combined with Artificial Intelligence/Machine Learning to determine the timing and degree of disease emergence in hiPSC disease models. In the preferred embodiment, continuous phenotypic monitoring is performed throughout differentiation using fully automated live cell culture instruments that include internal cell culture incubators, internal electrophysiologic sensors, and/or internal microscopes and associated lenses and filters for brightfield, phase, fluorescence and other morphologic and biomarker signal detection. In one embodiment, standard hiPSC culture and differentiation is performed by skilled laboratory personnel without automated instrumentation. Artificial Intelligence/Machine Learning applied to these phenotypic monitoring data aids in the identification of the first timepoint at which disease emerges in specific cell sub-populations during differentiation.

Monitoring for disease in differentiating hiPSCs using high resolution phenotypic detection methods and single cell sequencing technologies identifies the specific cell type and specific timepoint at which a pathogenic DNA mutation causes disease. For example, if a disease phenotype in a sub-population of cells is detected on day 10 of hiPSC differentiation, then those genomic regions that became newly active or inactive in that cell type on day 10 will contain DNA variants with the highest probability of pathogenicity.

When a disease phenotype is detected in differentiating hiPSCs, then single cell genetic studies are performed immediately before, during, and after the timepoint when disease is first detected. Because hiPSC disease models are composed of mixed cell populations, single cell genetic studies combined with high resolution cellular phenotyping methods identify the specific cell type and associated DNA mutation(s) causing a disease phenotype. In the preferred embodiment, both single cell RNA-seq (gene expression) and single cell single cell assay for transposase-accessible chromatin (ATAC)-seq (epigenome “open chromatin”) are employed to determine: (a) disease-specific gene expression in each cellular subtype of a disease model, and (b) open or closed epigenomic regions that are associated with disease emergence. In one embodiment, only single cell RNA-seq is performed. In another embodiment, only ATAC-seq is performed. In another embodiment, any single cell genetic detection method is used to assess single cell genetic activity that may include single cell chromosome conformation capture (e.g. scHi-C), single cell chromatin immunoprecipitation sequencing (scChIP-seq), or any other single cell genetic assay.

Artificial Intelligence/Machine Learning analysis (270) of single cell sequencing dynamics around the period of disease emergence in hiPSC disease models narrows the list of DNA variants to cell type-specific genomic regions whose change in activity correlate with disease emergence. These data can then be used to determine the specific DNA mutation(s) that have the highest correlation with disease in a specific cell type (280).

### Indications.

Cardiovascular Diseases, including cardiomyopathies (Sun N, et al. Patient-specific induced pluripotent stem cells as a model for familial dilated cardiomyopathy. Science Translational Medicine. 2012; 4:130ra47. Lan F, et al. Abnormal calcium handling properties underlie familial hypertrophic cardiomyopathy pathology in patient-specific induced pluripotent stem cells. Cell Stem Cell. 2013; 12:101-13. Each is incorporated by reference in its entirety for all purposes), Brugada syndrome (Liang P, et al. Patient-specific and genome-edited induced pluripotent stem cell-derived cardiomyocytes elucidate single cell phenotype of Brugada Syndrome. J Am Coll Cardiol. 2016; 68:2086-2096, which is incorporated by reference in its entirety for all purposes) and other arrhythmias, hereditary angioedema, Tetralogy of Fallot (Grunert M, et al. Induced pluripotent stem cells of patients with Tetralogy of Fallot reveal transcriptional alterations in cardiomyocyte differentiation. Scientific Reports. 2020; 10, which is incorporated by reference in its entirety for all purposes), great vessel transposition, other congenital diseases. Differentiation of hiPSCs includes cardiomyocytes (Burridge, P. W., Matsa, E., Shukla, P., et al. (2014). Chemically defined generation of human cardiomyocytes. Nat Methods 11, 855-860, which is incorporated by reference in its entirety for all purposes). Chemically defined generation of human cardiomyocytes. Nat Methods 11, 855-860, which is incorporated by reference in its entirety for all purposes), endothelial cells, smooth muscle cells, cardiac fibroblasts, multicellular 2D beating organoids (Myers, F. B., Silver, J. S., Zhuge, Y., et al. (2013). Robust pluripotent stem cell expansion and cardiomyocyte differentiation via geometric patterning. Integr Biol 5, 1495-1506, which is incorporated by reference in its entirety for all purposes), or multicellular 3D organoids and engineered heart tissues that may include blood vessels. Disease phenotypic monitoring during and after differentiation may include live cell microscopy, immunophenotyping, flow cytometry, FACS, electrophysiologic measurements, calcium dynamics, contraction and sarcomeric measurements, migration, angiogenic vessel formation, morphology and function.

Blood Diseases, including hemophilias (Jia B, et al. Modeling of hemophilia A using patient-specific induced pluripotent stem cells derived from urine cells. Life Sciences. 2014; 108:22-29. Rose M, et al. Endothelial cells derived from patients' induced pluripotent stem cells for sustained factor VIII delivery and the treatment of hemophilia A. Stem Cells Translational Medicine. 2020; 9. Each is incorporated by reference in its entirety for all purposes) and factor deficiency coagulopathies, sickle cell anemia (Park S, et al. A comprehensive, ethnicially diverse library of sickle cell disease-specific induced pluripotent stem cells. Stem Cell Reports. 2017; 8:1076-1085, which is incorporated by reference in its entirety for all purposes), aplastic anemia (Melguizo-Sanchis D, et al. iPSC modeling of severe aplastic anemia reveals impaired diferentiation and telomere shortening in blood progenitors. Cell Death & Disease. 2018; 9, which is incorporated by reference in its entirety for all purposes), Diamond-Blackfan anemia (Doulatov S, et al. Drug discovery for Diamond-Blackfan anemia using reprogrammed hematopoietic progenitors. Science Translational Medicine. 2017; 9, which is incorporated by reference in its entirety for all purposes), thalassemia syndromes Song B, et al. Improved hematopoietic differentiation efficiency of gene-corrected beta-thalassemia induced pluripotent stem cells by CRISPR/Cas9 system. Stem Cells Dev. 2014; 24, which is incorporated by reference in its entirety for all purposes). Differentiation of hiPSCs includes hematopoietic progenitor cells, red blood cells, white blood cells, progenitor bone marrow tissues, hepatocytes, endothelial cells, liver tissue, splenic tissue, lymphoid tissue. Disease phenotypic monitoring during and after differentiation may include live cell microscopy, immunophenotyping, flow cytometry, FACS, hemoglobin assays, coagulation assays, oxygen carrying capacity.

Neurologic diseases, including neurofibromatosis (Wegscheid M L, et al. Human stem cell modeling in neurofibromatosis type 1 (NF1). Experimental Neurology. 2018; 299:270-280, which is incorporated by reference in its entirety for all purposes), Huntington's Disease (The HD iPSC Consortium. Induced pluripotent stem cells from patients with Huntington's Disease show CAG-repeat-expansion-associated phenotypes. Cell Stem Cell. 2012; 11:264-278, which is incorporated by reference in its entirety for all purposes), spinal muscular atrophy (Corti S, et al. Genetic correction of human induced pluripotent stem cells from patients with spinal muscular atrophy. Science Translational Medicine. 2012; 4:165ra162, which is incorporated by reference in its entirety for all purposes), genetic epilepsies (Tidball A M and Parent J M. Exciting cells: Modeling genetic epilepsies with patient-derived induced pluripotent stem cells. Stem Cells. 2015; 34:27-33, which is incorporated by reference in its entirety for all purposes), Charcot-Marie-Tooth Disease (Saporta M A, et al. Axonal Charcot-Marie-Tooth disease patient-derived motor neurons demonstrate disease-specific phenotypes including abnormal electrophysiological properties. Experimental Neurology. 2015; 263:190-199, which is incorporated by reference in its entirety for all purposes). Disease models include hiPSC-derived neurons, glial cells, astrocytes, motor neurons, models of spinal chord biogenesis, brain organoids (Di Lullo E and Kriegstein A R. The use of brain organoids to investigate neural development and disease. Nature Reviews Neuroscience. 2017; 18:573-584, which is incorporated by reference in its entirety for all purposes), and neuron-muscle cell co-cultures. Disease phenotypic monitoring during and after differentiation may include live cell microscopy, immunophenotyping, flow cytometry, FACS, electrophysiologic measurements, neurotransmitter assays, cell-to-cell signaling assays, cellular migration and death assays.

Respiratory diseases, including cystic fibrosis (Firth A L, et al. Functional gene correction for cystic fibrosis in lung epithelial cells generated from patient hiPSCs. Cell Reports. 2015; 12:1385-1390, which is incorporated by reference in its entirety for all purposes). Disease models include airway cells in bronchial and bronchiolar epithelium and bronchial glands (basal, secretory, ciliated and neuroendocrine cells), alveolar unit ells, pulmonary vascular cells, multicellular 2D and 3D lung and pancreatic organoids. Disease phenotypic monitoring during and after differentiation may include live cell microscopy, immunophenotyping, flow cytometry, FACS, electrophysiologic measurements, cellular secretory and ciliated health, cystic fibrosis transmembrance conductance regulator (CFTR) protein function, chloride measurements, pancreatic cellular function.

Endocrine diseases, including acromegaly, growth hormone deficiency, hypophosphatemia, multiple endocrine neoplasia, congenital adrenal hyperplasia. Disease models include all cell types and tissues of the endocrine system, including pituitary, adrenal, pancreas, thyroid, parathyroid, pineal, testes, ovaries, hypothalamus. Disease phenotypic monitoring during and after differentiation may include live cell microscopy, immunophenotyping, flow cytometry, FACS, electrophysiologic measurements, cellular secretory health, hormone generation and function.

Musculoskeletal diseases, including muscular dystrophies (Maffioletti S M, et al. Three-dimensional human hiPSC-derived artificial skeletal muscles model muscular dystrophies and enable multilineage tissue engineering. Cell Reports. 2018; 23:899-908. Smith A S T, et al. Muscular dystrophy in a dish: engineered human skeletal muscle mimetics for disease modeling and drug discovery. Drug Discovery Today. 2016; 21:1387-1398. Each is incorporated by reference in its entirety for all purposes) such as Duchenne muscular dystrophy (Shoji, E., Sakurai, H., Nishino, T., et al. (2015). Early pathogenesis of Duchenne muscular dystrophy modelled in patient-derived human induced pluripotent stem cellsw. Sci Rep 5, 12831, which is incorporated by reference in its entirety for all purposes). Disease models include skeletal and cardiac muscle cellular lineages and tissues. Disease phenotypic monitoring during and after differentiation may include live cell microscopy, immunophenotyping, flow cytometry, FACS, electrophysiologic measurements, calcium dynamics, contraction and sarcomeric measurements, myofibril measurements, cellular migration, angiogenic vessel formation, morphology and function.

Gastro-intestinal diseases, including Hirschprung's Disease (Fattahi F, et al. Deriving human ENS lineages for cell therapy and drug discovery in Hirschsprung disease. Nature. 2016; 531:105-109. Lai F P L, et al. Correction of Hirschsprung-associated mutations in human induced pluripotent stem cells via clustered regularly interspaced short palindromic repeats/Cas9, restores neural crest cell function. Gastroenterology. 2017; 153:139-153. Each is incorporated by reference in its entirety for all purposes). Disease models include neural crest progenitor cells and tissues, neuronal cells and tissues, small and large intestinal cells and tissues. Disease phenotypic monitoring during and after differentiation may include live cell microscopy, immunophenotyping, flow cytometry, FACS, electrophysiologic measurements, calcium dynamics, contraction and sarcomeric measurements, myofibril measurements, migration, angiogenic vessel formation, morphology and function, neurotransmitter assays, cell-to-cell signaling assays, cellular migration and death assays.

Dermatologic diseases, including Ehlers-Danlos syndrome, albinism, ectodermal dysplasias (Shalom-Feuerstein R, et al. Impaired epithelial differentiation of induced pluripotent stem cells from ectodermal dysplasia-related patients is rescued by the small compound APR-246/PRIMA-1 MET. PNAS. 2013; 110:2152-2156, which is incorporated by reference in its entirety for all purposes), Tuberous sclerosis, Incontinentia pigmenti, Ichthyoses. Disease models include keratinocytes, melanocytes, Langerhans cells, Merkel cells, and 2D and 3D multicellular organoids and tissue mimics. Disease phenotypic monitoring during and after differentiation may include live cell microscopy, immunophenotyping, flow cytometry, FACS, melanin health and dynamics, assays of aging, collagen elasticity.

Protein, lipid and lysosomal disorders, including Alpha-1 antitrypsin deficiency (Kaserman J E, et al. A highly phenotyped open access repository of alpha-1-antitrypsin deficiency pluripotent stem cells. Stem Cell Reports. 2020; 15:242-255. Wilson A A, et al. Emergence of a stage-dependent human liver disease signature with directed differentiation of alpha-1-antitrypsin-deficient iPS cells. Stem Cell Reports. 2015; 4:873-885. Each is incorporated by reference in its entirety for all purposes), Gaucher disease Borger D K, et al. Applications of hiPSC-derived models of Gaucher disease. Ann Transl Med. 2015; 3:295, which is incorporated by reference in its entirety for all purposes), Fabry Disease (Chanana A M, et al. Human induced pluripotent stem cell approaches to model inborn and acquired metabolic heart diseases. Current Opinion in Cardiology. 2016; 31:266-274. Itier J-M, et al. Effective clearance of GL-3 in a human hiPSC-derived cardiomyocyte model of Fabry disease. Journal of Inherited Metabolic Disease. 2014; 37:1013-1022. Each is incorporated by reference in its entirety for all purposes), Niemann-Pick Disease (Maetzel D, et al. Genetic and chemical correction of cholesterol accumulation and imparied autophagy in hepatic and neural cells derived from Niemann-Pick Type C patient-specific iPS cells. Stem Cell Reports. 2014; 2:866-880, which is incorporated by reference in its entirety for all purposes), Pompe Disease (Chanana A M, et al. Human induced pluripotent stem cell approaches to model inborn and acquired metabolic heart diseases. Current Opinion in Cardiology. 2016; 31:266-274. Huang H-P, et al. Human Pompe disease-induced puripotent stem cells for pathogenesis modeling, drug testing and disease marker identification. Human Molecular Genetics. 2011; 20:4851-4864. Each is incorporated by reference in its entirety for all purposes), Familial Hypercholesterolemia (Cayo M A, et al. JD induced pluripotent stem cell-derived hepatocytes faithfully recapitulate the pathophysiology of familial hypercholesterolemia. Hepatology. 2012; 56:2163-2171, which is incorporated by reference in its entirety for all purposes). Disease models include all cell types, tissues and organs specifically affected by these protein, lipid and lysosomal disorders. As many of the protein, lipid and lysosomal orders affect multiple organs, disease models may also include embryonic differentiation (embryoids or embryoid bodies) that contain cells derived from all three embryonic germ layers. Disease phenotypic monitoring during and after differentiation likewise includes cell type-specific, tissue-specific and organ-specific assays of health and disease tailored to the specific disease etiology.

Cancer, including lymphoblastic and myeloid leukemias (Papapetrou E P. Modeling leukemia with human induced pluripotent stem cells. Cold Spring Harb Perspect Med. 2019; 9:a034868, which is incorporated by reference in its entirety for all purposes), lymphomas, neuroblastoma, glioblastoma, Ewing's sarcoma, osteosarcoma (Lin Y-H, et al. Osteosarcoma: Molecular Pathogenesis and hiPSC Modeling. Trends in Molecular Medicine. 2017; 23:737-755, which is incorporated by reference in its entirety for all purposes), Wilms tumor, rhabdomyosarcoma, retinoblastoma, spinal cord tumors, Li-Fraumeni syndrome (Zhou R, et al. Li-Fraumeni Syndrome disease model: A platform to develop precision cancer therapy targeting oncogenic p53. Trends in Pharmacological Sciences. 2017; 38:908-927, which is incorporated by reference in its entirety for all purposes). Disease models include all cell types, tissues and organs specifically affected by these cancers. As many cancers may affect multiple organs, disease models may also include embryonic differentiation (embryoids or embryoid bodies) that contain cells derived from all three embryonic germ layers. To mimic the potential for stem cell-based etiology of cancers, hiPSC-derived cells and tissues may be repogrammed back to undifferentiated hiPSCs using Yamanaka factor methods, and then re-differentiated to the cell type or tissue of interest. Multiple cycles of differentiation/repogramming may be required to elicit the cancer phenotype. Disease phenotypic monitoring during and after differentiation likewise includes cell type-specific, tissue-specific and organ-specific assays of health and disease tailored to the specific cancer. In general this includes assays for cellular invasion, migration, morphology, immunophenotyping, nuclear-to-cytoplasm ratios, cytogenetics and/or DNA mutation monitoring, mitosis and cellular division. To elicit the cancer phenotype in hiPSC disease models, external stressors such as ionizing radiation may be applied.

Immunological diseases, including juvenile arthritis, Type 1 diabetes (Leite N C, et al. Modeling Type 1 Diabetes in vitro using human pluripotent stem cells. Cell Reports. 2020; 32, which is incorporated by reference in its entirety for all purposes), and severe combined immunodeficiency (Chang, C.-W., Lai, Y.-S., Westin, E., and Khodadadi-Jamayran, A. (2015). Modeling of human severe combined immunodeficiency correction by CRISPR/Cas9-enhanced gene targeting. Cell Reports 12, 1668-1677, which is incorporated by reference in its entirety for all purposes). Differentiation of hiPSCs includes hematopoietic progenitor cells, white blood cells including CD8 and CD4 immune cells, progenitor bone marrow tissues, hepatocytes, pancreatic cells, endothelial cells, liver tissue, splenic tissue, lymphoid tissue. Disease phenotypic monitoring during and after differentiation may include live cell microscopy, immunophenotyping, flow cytometry, FACS, immunoglobulin measurements.

Syndromic diseases, including Fragile X syndrome (Sheridan S D, et al. Epigenetic charaterization of the FMR1 gene and aberrant neurodevelopment in humnan induced pluripotent stem cell models of Fragile X syndrome. PloS One. 2011; 6:e26203, which is incorporated by reference in its entirety for all purposes), Prader-Willi and Angelman syndromes (Chamberlain S J, et al. Induced pluripotent stem cell models of the genomic imprinting disorders Angelman and Prader-Willi syndromes. PNAS. 2010; 107, which is incorporated by reference in its entirety for all purposes), Hutchinson-Gilford Progeria (Liu G-H, et al. Recapitulation of premature ageing with hiPSCs from Hutchinson-Gilford progeria syndrome. Nature. 2011; 472:221-225. Zhang J, et al. A human hiPSC model of Hutchinson Gilford Progeria reveals vascular smooth muscle and mesenchymal stem cell defects. Cell Stem Cell. 2011; 8:31-45. Each is incorporated by reference in its entirety for all purposes), and Rett syndrome (Marchetto M D N, et al. A model for neural development and treatment of Rett Syndrome using human induced pluripotent stem cells. Cell. 2010; 143:527-539, which is incorporated by reference in its entirety for all purposes). Disease models include all cell types, tissues and organs specifically affected by these syndromic diseases. As many of the syndromic diseases affect multiple organs, disease models may also include embryonic differentiation (embryoids or embryoid bodies) that contain cells derived from all three embryonic germ layers. Disease phenotypic monitoring during and after differentiation likewise includes cell type-specific, tissue-specific and organ-specific assays of health and disease tailored to the specific disease etiology.

### Applications

Although advancements such as amniocentesis, sonography, protein biomarkers, and cell-free DNA testing have enabled increasingly sensitive detection of some fetal genetic diseases such as Trisomy 21 during pregnancy, the vast majority of rare diseases are still inadequately diagnosed and treated in early life. This is because widespread use of fetal tissues in drug discovery pipelines is logistically challenging, limited in quantity, and ethically challenging. Rapid and high throughput hiPSC models of human development address these bottlenecks for discovering the genetic causes of early life diseases and, importantly, enable temporal detection of the effect of genome on disease phenotype.

The Discovery System can be used to overcome this bottleneck using continuous phenotypic monitoring throughout the hiPSC differentiation period combined with Artificial Intelligence/Machine Learning to determine the timing and degree of disease emergence in hiPSC disease models. Starting at the embryonic stage, hiPSCs derived from a child with disease can be differentiated to near-neonatal age tissues in vitro that mimic the child's own development in utero, including his/her specific genotype and phenotype, and will therefore capture the specific timepoint at which the child's disease emerged during his/her development. This can identify the specific DNA mutations and gene expression changes that lead to emergence of disease in the developing fetus. No other current platform, biological or genetic, can perform such a function, and the information from patient-specific hiPSC experiments can be used to (1) identify new drug targets, and (2) identify targeted drug therapies that can be used to treat rare diseases in children.

Upon detection by phenotypic monitoring of disease phenotype in hiPSC disease models, single cell sequencing assays, including single cell RNA-seq and/or single cell ATAC-seq, are performed before, during and after the time point when disease is first detected, as described in (300). DNA variants that occur in genomic regions showing dynamic changes (gene expression, open chromatin) at the same time as disease emergence in hiPSC disease models have the highest likelihood of being true pathogenic mutations. DNA mutations such as substitutions, insertions and deletions are those that occur within protein-coding genes (exons or introns) and lead to amino acid changes, within non-coding genes such as long non-coding RNAs or microRNAs, or in regulatory regions such as gene promoters, enhancers, and insulators that induce expression changes in downstream genes (The GTEx Consortium (2020). The GTEx Consortium atlas of genetic regulatory effects across human tissues. Science 369, 1318-1330, which is incorporated by reference in its entirety for all purposes). Larger mutations such as chromosomal inversions and large deletions may also occur across multiple genes and are typically detected through cytogenetic methods. Gene editing methods such as CRISPR are then used to correct these mutations in patient-specific hiPSCs, which are then differentiated to the cell type or tissue of interest with continuous phenotypic monitoring as before. In the case of gene expression changes associated with disease emergence, alteration of the activity or expression of these identified genes in hiPSC disease models followed by repeat disease modeling to determine the degree of disease resolution can be performed with methods that may include RNA interference (RNAi), short hairpin RNA (shRNA), and CRISPR. Amelioration of the disease phenotype in corrected hiPSC models then validate that the candidate DNA mutation or gene expression change is truly pathogenic.

Once a DNA mutation or gene expression change has been experimentally validated, drug discovery can then be initiated using the validated hiPSC disease models described above. This may involve high throughput screens with small molecules and/or existing drugs applied to the disease models to look for reduction of disease. Other interventions may include gene therapies for correction of the DNA mutation directly, or allele specific oligonucleotides and RNA interference for targeting RNA molecules. Antibody therapies may also be utilized to target disease-causing proteins that result from DNA mutations.

Expanding orphan indications for existing drugs. Most medications used in pediatric patients are not FDA approved for use in this patient population. Off-label use is the mainstay of therapy in pediatric patients even though many lack scientific support of efficacy or safety, and some have only anecdotal or case report data (Rumore, M. M. (2016). Medication repurposing in pediatric patients: teaching old drugs new tricks. J Pedatr Pharmacol 21, 36-53, which is incorporated by reference in its entirety for all purposes). Even with passage of the US Orphan Drug Act in 1983, two major impediments to approved pediatric indications remain: challenges in obtaining fetal and pediatric tissues for genetic study, and challenges in conducting clinical trials. Many existing drugs are instead re-positioned for indications that target a different age- or biomarker-based subset of a rare disease that the drug had already been approved to treat. Using drugs with known safety profiles streamlines the clinical trial by bypassing Phase I. Re-purposing existing drugs is therefore a viable, risk-managed strategy for pharmaceutical companies developing pediatric orphan drugs with potentially lower costs and shorter timelines.

The majority (56%) of FDA-approved pediatric orphan indications between 2010 and 2018 were for drugs already approved to treat at least one other disease. For example, adalimumab (Humira) was approved for several common autoimmune conditions in adults, such as rheumatoid arthritis, at the time it received a pediatric orphan indication for juvenile idiopathic arthritis in children ages 2 to 3 years in 2014, which is 6 years after it received an orphan indication for this disease in children 4 years and older (Kimmel, L., Conti, R. M., Volerman, A., et al. (2020). Pediatric orphan drug indications: 2010-2018. Pediatrics 145, e20193128, which is incorporated by reference in its entirety for all purposes). A second example, lumacaftor-ivacaftor (Orkambi) was initially approved in 2015 as an orphan drug for patients with cystic fibrosis who were aged ≥12 years with the F508del CFTR gene mutation and subsequently gained 2 additional orphan indications for patients with cystic fibrosis with this mutation who were aged 6 to 11 years and aged 2 to 5 years. Using this Discovery System (300) and appropriate patient-derived hiPSCs can mitigate the risks in obtaining and/or expanding orphan indications for existing drugs.

Drug discovery utilizing hiPSC disease models will also expedite early life drug discovery and development by providing a human-based fetal model system of disease. Once candidate drugs or interventions are identified that ameliorate or “cure” disease in hiPSC disease models, then testing can proceed to animal models (e.g. rodent, swing, non-human primate) as necessary for further determination of drug efficacy, specificity and toxicity. After pre-clincial testing, drugs that have demonstrated adequate efficacy and safety in cellular and animal models can then proceed to human clinical trials.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: Genetically Un-Defined Diseases

One group of rare diseases with few treatment options are pediatric cardiomyopathies that weaken the heart's ability to pump blood. Cardiomyopathies result in some of the worst pediatric cardiology outcomes; nearly 40% of children who present with symptomatic cardiomyopathy undergo a heart transplantation or die within the first 2 years after diagnosis (Lipshultz, S. E., Law, Y. M., Asante-Korang, A., et al. (2019). Cardiomyopathy in children: Classification and diagnosis: A scientific statement from the American Heart Association. Circulation 140, e9-e68, which is incorporated by reference in its entirety for all purposes). The percentage of children with cardiomyopathy who underwent a heart transplantation has not declined over the past 10 years, and cardiomyopathy remains the leading cause of transplantation for children >1 year of age. Causes are established in very few children with cardiomyopathy, yet genetic causes are likely to be present in most (Lipshultz, S. E., Law, Y. M., Asante-Korang, A., et al. (2019). Cardiomyopathy in children: Classification and diagnosis: A scientific statement from the American Heart Association. Circulation 140, e9-e68, which is incorporated by reference in its entirety for all purposes). The incidence of pediatric cardiomyopathy is ˜1 per 100,000 children. This is comparable to the incidence of childhood cancers as lymphoma, Wilms tumor, and neuroblastoma. Over $1 billion annually in inpatient charges alone for pediatric heart failure in the U.S. (Nandi, D., and Rossano, J. W. (2015). Epidemiology and cost of heart failure in children. Cardiol Young 25, 1460-1468, which is incorporated by reference in its entirety for all purposes). In 2019, there were >500 pediatric heart transplants in the U.S. at a cost of $400 million (Godown, J., Thurm, C., Hall, M., et al. (2018). Changes in pediatric heart transplant hospitalization costs over time. Transplantation 102, 1762-1767, which is incorporated by reference in its entirety for all purposes).

A simplified hiPSC model system of pediatric cardiomyopathies has already yielded a novel gene expression change and drug target (Wilson, K. D., Ameen, M., Guo, H., et al. (2020). Endogenous retrovirus-derived lncRNA BANCR promotes cardiomyocyte migration in humans and non-human primates. Dev Cell 54, 694-709, which is incorporated by reference in its entirety for all purposes). Using the Discovery System, which includes Artificial Intelligence/Machine Learning and single cell genomics as well as hiPSC disease models, more sensitive and high throughput drug discovery is now possible. In this example, hiPSC lines previously generated from children with a history of cardiomyopathy are acquired from the California Institute of Regenerative Medicine (CIRM) Biobank, which is currently maintained by Fuji Film/Cellular Dynamics (https://www.fujifilmedi.com/search-cirm/). The genetic mutations causing cardiomyopathies in these children are unknown. In the Discovery System, each hiPSC line will be subjected to whole genome sequencing and a list of candidate DNA variations for each individual cell line is generated. We expect that in most cases the pathogenic DNA mutation will be unclear due to inadequate genomic annotation and/or numerous other potentially pathogenic muations that are also present in each individual. Cytogenetic methods will be employed to rule out large chromosomal abberations in hiPSC lines that can explain the cardiomyopathies in these children.

To identify the true pathogenic DNA mutation(s) and/or gene expression change(s) causing cardiomyopathies in these children and discover potential drug targets, each hiPSC line will be micropatterned onto tissue culture plates such that hiPSCs are organized and arrayed as circular clusters of cells. Wilson et al. showed increased cardiomyocyte migration in children with dilated cardiomyopathies, and therefore increased cellular migration will be a key phenotypic measure of disease in hiPSC models. Over the course of 2-4 weeks, micropatterned hiPSCs will be differentiated into cardiac fetal tissues using published cardiac differentiation protocols and continuously monitored with live cell microscopy for evidence of increased cellular migration. Artificial Intelligence/Machine Learning will analyze the continuous imaging data of differentiating hiPSC micropatterns, and will aid in the detection of the first sign of disease. Note that Artificial Intelligence/Machine Learning will have already been trained on normal wildtype hiPSC models of cardiac development. Negative control (wildtype) hiPSC micropattern models may also be run in parallel with disease hiPSC models, as needed.

Once disease emergence (increased migration in cardiomyocytes) is detected by live cell microscopy and Artificial Intelligence/Machine Learning in hiPSC disease models, the experiment will then be repeated for each patient-specific hiPSC line and cells collected before and after the timepoint of disease emergence as determined previously. Disease emergence is expected to occur in precursor cardiovascular tissue when genetic regulators of heart development are most active. Single cell RNA-seq and single cell ATAC-seq libraries are then prepared from genomic isolates of timepoint-specific and patient-specific hiPSC micropatterns. Note that each differentiated micropattern contains multi-lineage cell types (eg. fibroblast, smooth muscle, cardiomyocyte, and vascular precursor cells). After sequencing of scRNA and scATAC prepared libraries, Artifical Intelligence/Machine Learning algorithms will assist in the identification of unique DNA variants and gene expression changes that become active or inactive at the timepoint of phenotypic disease emergence in hiPSC disease models. In some cases the pathogenic DNA mutations or gene expression change may be active in non-cardiomyocyte cell types such as smooth muscle or endothelial cells that comprise the micropatterns in addition to cardiomyocytes. However, because most cardiomyopathies are a disease of cardiomyocytes, it is expected that single cell genomic studies will identify DNA mutations and/or gene expression changes causing disease in the cardiomyocyte fraction of hiPSC micropatterns, and therefore the focus will be on those cell types.

Examples of mutations may include DNA mutations within protein-coding genes that begin to be transcribed (expressed) at the timepoint of disease emergence, as measured by scRNA-seq. DNA mutations may also occur in non-coding genes such as microRNAs and long noncoding RNAs. Finally, DNA mutations may occur in regulatory regions (eg. gene promoters and enhancers) that regulate expression of genes comprising critical molecular pathways. The activity or inactivity of key regulatory regions will be determined with scATAC-seq, a measure of open chromatin and therefore DNA accessibility and activity.

By determining both the patient-specific whole genome sequence and genome-wide RNA expression (the “transcriptome”) at specific timepoints during cardiac differentiation, the Discovery System is also able to detect expressed quantitative trait loci (eQTL) in patient-specific hiPSC models that are correlated with emergence of cardiomyopathy. Chromosomal loci that explain variance in expression traits are called eQTLs. Importantly, the abundance of a gene transcript can be directly modified by DNA mutations or SNPs in regulatory gene elements such as promoters, enhancers, insulators or untranslated regions (UTRs) (Zhu Z., et al. Integration of summary data from GWAS and eQTL studies predicts complex trait gene targets. Nature Genetics. 2016; 48:481-487, which is incorporated by reference in its entirety for all purposes). By assaying gene expression and genetic variation simultaneously on a genome-wide basis, statistical genetic methods can be used to map the genetic factors that underpin cardiomyopathy emergence in quantitative levels of expression of many thousands of transcripts.

Once candidate pathogenic DNA mutations and gene expression changes are identifited by the Discovery System at the timepoint when disease emerges in patient-specific hiPSC models, gene editing experiments in patient-specific hiPSCs can be performed to “correct” (remove) the mutation or correct the aberrant gene expression change, as described earlier. hiPSC disease modeling experiments are then repeated with corrected hiPSC lines from each patient, with the expectation that disease (increased cardiomycyte migration) is no longer detectable. Alteration of the activity or expression level of genes in hiPSC disease models followed by repeat disease modeling to determine the degree of disease resolution can be performed with methods that may include RNA interference (RNAi), short hairpin RNA (shRNA), and CRISPR. These validated DNA mutations are then direct drug targets in addition to any aberrantly-expressed genes downstream of DNA mutations in gene regulatory regions, and can also be targeted for drug discovery.

Because cells differentiated from disease- and patient-specific hiPSCs exhibit disease processes at the single cell level, they are an attractive option for screening therapeutic compounds. High throughput screens of pharmaceuticals against cells differentiated from disease hiPSCs have allowed for rapid assessment of efficacy and toxicity (Liang, P., Lan, F., Lee, A. S., et al. (2013). Drug screening using a library of human induced pluripotent stem cell-derived cardiomyocytes reveals disease-specific patterns of cardiotoxicity. Circulation 127, 1677-1691, which is incorporated by reference in its entirety for all purposes). Additionally, given the genetic heterogeneity underlying many common diseases for which treatments are ineffective due to unpredictable patient response, hiPSC disease models also present an opportunity to tailor therapies to the specific disease-causing DNA mutation or gene expression change. The identified compounds, or combinations of compounds, would then be broadly applicable to all patients carrying the same mutation. For these reasons, hiPSCs have elicited interest from the pharmaceutical industry in the hopes that research and development can be greatly streamlined. For example, cardiomyopathic disease-specific hiPSC lines will help expedite identification of drug candidates and accelerate the screening of toxic and off-target effects.

### Example 2: Genetically Defined Diseases

Even if the genetic cause of a child's rare disease is already known (“defined”), targeted treatments against the specific DNA mutation may not exist or, for various technical reasons, not possible. In fact, 95% of rare diseases have no treatment. Some examples of genetically defined rare diseases in children include:

Duchenne muscular dystrophy (1 in 3,500 male births; half of patients are deceased by age 25; cardiomyopathy is the leading cause of death)

Spinal muscular atrophy (1 in 10,000 live births; the most common genetic cause of mortality in infants)

Cystic fibrosis (1 in 3,000 Caucasian live births)

These diseases are known single-gene disorders (DMD, SMN1 and CFTR, respectively) in which there are hundreds, even thousands, of known DNA mutations. Some, but not all, mutations can markedly affect the mechanism and severity of disease. For this reason, pharmaceutical companies often develops specific drugs for each mutation (as in cystic fibrosis), a costly and lengthy process as discussed previously. The Discovery System can directly address both cost and time for these companies. To expedite acquisition of relevant hiPSC disease models for the Discovery System, a biobank of gene edited hiPSC lines each with a unique disease-causing DNA mutation can be generated. These lines can then be used for drug discovery or screening with existing drugs using the Discovery System.

Similar to cardiomyopathy applications in children, each of the above three diseases can be modeled in vitro with genetically defined hiPSCs. For each disease the differentiation protocols will be tailored to the specific disease and relevant cell/organ/tissue system. For example, hiPSCs from children with Duchenne muscular dystrophy will be differentiated into skeletal muscle lineages; hiPSCs from children with spinal muscular atrophy will be differentiated into motor neurons; hiPSCs from children with cystic fibrosis will be differentiated into lung epithelial tissues. In some cases simple differentiation protocols will suffice that yield homogenous cell cultures (e.g motor neurons), in other cases micropattern-derived fetal tissue models that utlize micropattern methods will yield more relavent tissue models (e.g. cystic fibrosis, muscular dystrophy).

As with the example of pediatric cardiomyopathy, differentiating hiPSC models will be continuously monitored for emergence of disease with the assistance of Artificial Intelligence/Machine Learning. For Duchenne muscular dystrophy, monitoring for disease emergence hiPSC-derived skeletal myotybes may include measurements of calcium ion influx and secretion of creatine kinase (Shoji, E., Sakurai, H., Nishino, T., et al. (2015). Early pathogenesis of Duchenne muscular dystrophy modelled in patient-derived human induced pluripotent stem cellsw. Sci Rep 5, 12831, which is incorporated by reference in its entirety for all purposes); for cystic fibrosis, monitoring for disease emergence in hiPSC-derived pancreatic or lung organoids may include measurements of chloride ion channel activity (Firth A L, et al. Functional gene correction for cystic fibrosis in lung epithelial cells generated from patient hiPSCs. Cell Reports. 2015; 12:1385-1390, which is incorporated by reference in its entirety for all purposes); for spinal muscular atrophy, monitoring for disease emergence in hiPSC-derived motor neurons may include measurements of neurite outgrowth (Corti, S., Nizzardo, M., Simone, C., et al. (2012). Genetic correction of human induced pluripotent stem cells from patients with spinal muscular atrophy. Science Translational Medicine 4, 165ra162, which is incorporated by reference in its entirety for all purposes).

When genetically defined hiPSCs (or gene edited hESCs) that carry known pathogenic mutations are employed in the Discovery System, then genetic studies (whole genome sequencing, single cell genetic studies) may not be necessary as the pathogenic mutation(s) are already known. However, important downstream gene expression changes may also exist that are directly regulated by a primary DNA mutation. For example, a pathogenic DNA mutation in a regulatory region (e.g. a gene's promoter or enhancer) may be causing aberrant expression of a nearby gene which then leads to disease in a patient. In this scenario, rather than develop drugs against the primary DNA mutation in the gene's regulatory region, it may be simpler to instead target the secondary aberrantly expressed gene (or its RNA transcript) in order to treat the disease. Therefore, in the preferred embodiment single cell RNA-seq and/or ATAC-seq to discover secondary molecular targets will be performed on genetically definied hiPSCs (and gene edited hESCs) for these three diseases.

This Discovery System identifies the DNA variants associated with disease emergence in hiPSC disease models and which are the true pathogenic mutations and gene expression changes underlying early life disease. Gene editing methods such as CRISPR are then used to remove or correct candidate mutation(s) in patient hiPSC lines, followed by repeat disease modeling to determine the degree of disease resolution.

Those skilled in the art will recognize, or be able to ascertain using no more than routine experimentation, many equivalents to the specific embodiments of the invention described herein. Such equivalents are intended to be encompassed by the following claims.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

1. A method, comprising the steps of: identifying a plurality of genomic mutations in a plurality of cells from a subject, wherein the subject has a genetic disease, and wherein the identifying is done by comparing the genomic sequence of the subject with the genetic disease to a genomic sequence of a healthy subject; obtaining a stem cell from the subject with the genetic disease; treating the stem cell with an agent that induces the stem cell to differentiate into an adult cell type or an adult tissue; comparing the stem cell during its differentiation to a stem cell from a healthy subject which is treated with the agent to induce differentiation to the adult cell type or an adult tissue; and identifying a phenotype of the stem cell from the subject with the genetic disease that is different from the phenotype of the stem cell from the healthy subject.

2. The method of claim 1, wherein the stem cell is an induced pluripotent stem cell.

3. The method of claim 1, wherein the stem cell is an embryonic stem cell.

4. The method of claim 1, wherein the stem cell is an adult stem cell.

5. The method of claim 1, wherein the genetic disease is a cardiovascular disease, a blood disease, a neurological disease, a gastrointestinal disease, a dermatological disease, or an immunological disease.

6. The method of claim 1, wherein the cardiovascular disease is a cardiomyopathy.

7. The method of claim 1, wherein the respiratory disease is a cystic fibrosis.

8. The method of claim 1, wherein the musculoskeletal disease is a muscular dystrophy.

9. The method of claim 1, wherein the adult cell type or tissue is a cardiomyocyte.

10. The method of claim 1, wherein the adult cell type or tissue is a motor neuron.

11. The method of claim 9, wherein the phenotype is an abnormal migration in cardiomyocytes.

12. The method of claim 9, wherein the phenotype is an abnormal sarcomere in a cardiomyocyte.

13. The method of claim 9, wherein the phenotype is an abnormal calcium ion influx in a cardiomyocyte.

14. The method of claim 9, wherein the phenotype is an abnormal contraction in a cardiomyocyte.

15. The method of claim 9, wherein the phenotype is an abnormal electrophysiology in a cardiomyocyte.

16. (canceled)

17. (canceled)

18. (canceled)

19. (canceled)

20. (canceled)

21. (canceled)

22. (canceled)

23. (canceled)

24. (canceled)

25. The method of claim 1, further comprising the step of identifying a specific time point during the differentiation of the stem cell when the genetic disease first emerges in a differentiating stem cell from the subject with the genetic disease.

26. The method of claim 1, further comprising the step of identifying the cell type causing the genetic disease at the time the phenotypic change occurs.

27. The method of claim 1, wherein the adult cell type or tissue is a liver tissue.

28. The method of claim 1, wherein treating, comparing and identifying steps are automated.

29. A method, comprising the steps of: identifying a plurality of genomic mutations in a plurality of cells from a pediatric subject, wherein the subject has a genetic disease, and wherein the identifying is done by comparing the genomic sequence of the pediatric subject with the genetic disease to a genomic sequence of a healthy subject; obtaining a stem cell from the pediatric subject with the genetic disease; treating the stem cell with an agent that induces the stem cell to differentiate into a mature cell type or a mature tissue; comparing the stem cell during its differentiation to a stem cell from a healthy pediatric subject which is treated with the agent to induce differentiation to the mature cell type or the mature tissue; and identifying a phenotype of the stem cell from the pediatric subject with the genetic disease that is different from the phenotype of the stem cell from the healthy pediatric subject.
